(19) United States
(12) Patent Application Publication
(10) Pub. No.: US 2020/0010571 A1
(43) Pub. Date: Jan. 9, 2020

(54) Bispecific Anti-TNF-Alpha/IL-17A Antibodies and Anti-TNF-Alpha Antibodies and Methods of Their Use

(71) Applicant: Janssen Biotech, Inc., Horsham, PA (US)

(72) Inventors: Mark Chiu, Paoli, PA (US); Jennifer Nemeth-Seay, Fort Washington, PA (US); Tatiana Ort, Horsham, PA (US); Fang Shen, Fort Washington, PA (US)

(21) Appl. No.: 16/567,646

(22) Filed: Sep. 11, 2019

Related U.S. Application Data
(63) Continuation of application No. 15/417,560, filed on Jan. 27, 2017, now abandoned.
(60) Provisional application No. 62/288,124, filed on Jan. 28, 2016.

(51) Int. Cl. C07K 16/46; A61K 39/395; A61K 45/06; C07K 16/24

(52) U.S. Cl. CPC C07K 16/468; A61K 39/3955; A61K 2039/505; C07K 16/241; C07K 16/244; A61K 45/06

(57) ABSTRACT

The present invention relates to bispecific anti-TNF-α/IL-17A antibodies and anti-TNF-α antibodies, polynucleotides encoding the antibodies or fragments, and methods of making and using the foregoing.

### CROSS-REFERENCE TO RELATED APPLICATIONS

This application is a Continuation of U.S. application Ser. No. 15/417,560, filed 17 Jan. 2017, currently pending, which claims the benefit of U.S. Provisional Application Ser. No. 62/288,124 filed 28 Jan. 2016, the entire contents of which are incorporated herein by reference.

### SEQUENCE LISTING

This application contains a Sequence Listing submitted via EFS-Web, the entire content of which is incorporated herein by reference. The ASCII text file, created on 11 September 2019, is named JBI5080USCNT1SEQLIST.txt and is 52,176 bytes in size.

### FIELD OF THE INVENTION

The present invention relates to bispecific anti-TNF-α/IL-17A antibodies, anti-TNF-α antibodies, polynucleotides encoding the antibodies or fragments, and methods of making and using the foregoing.

### BACKGROUND OF THE INVENTION

Tumor necrosis factor-α (TNF-α) is a multifunctional pro-inflammatory cytokine. TNF-α triggers pro-inflammatory pathways that result in tissue injury, such as degradation of cartilage and bone, induction of adhesion molecules, induction of pro-coagulant activity on vascular endothelial cells, an increase in the adherence of neutrophils and lymphocytes, and stimulation of the release of platelet activating factor from macrophages, neutrophils and vascular endothelial cells.

Interleukin-17A (IL-17A) is an inflammatory cytokine produced by Th17 T cells. IL-17A may exist either as a homodimer or as a heterodimer complexed with its homolog IL-17F to form heterodimeric IL-17A/F. IL-17A is involved in the induction of pro-inflammatory responses and induces or mediates expression of a variety of other cytokines and mediators including TNF-α, IL-6, IL-8 (CXCL8), IL-Iβ, granulocyte colony-stimulating factor (G-CSF), prostaglandin E2 (PGE2), IL-10, IL-12, leukemia inhibitory factor, stromely sin, and nitric oxide.

Although biologic therapeutics that specifically bind to IL-17A or TNF-α have been produced, there remains a need for improved anti-inflammatory drugs that can effectively neutralize the activity of both IL-17A and TNF-α for the treatment of inflammatory and autoimmune diseases, for example rheumatoid arthritis, in which a significant portion of patients still do not respond adequately to therapy.

### BRIEF SUMMARY OF THE INVENTION

The invention provides for an isolated bispecific anti-tumor necrosis factor (TNF-α)/interleukin-17A (IL-17A) antibody comprising a first domain specifically binding TNF-α and a second domain specifically binding IL-17A, wherein the first domain comprises a heavy chain complementarity determining region (HCDR) 1, a HCDR2, a HCDR3, a light chain complementarity determining region (LCDR) 1, a LCDR2 and a LCDR3 of SEQ ID NOs: 15, 16, 17, 18, 19 and 20, respectively, and the second domain comprises the HCDR1, the HCDR2, the HCDR3, the LCDR1, the LCDR2 and the LCDR3 of SEQ ID NOs: 21, 22, 23, 24, 25 and 26, respectively.

The invention provides for an isolated bispecific anti-TNF-α/IL-17A antibody comprising

the HC1 and the LC1 of SEQ ID NOs: 5 and 6, respectively, and the HC2 and the

LC2 of SEQ ID NOs: 8 and 9, respectively; or

the HC1 and the LC1 of SEQ ID NOs: 7 and 6, respectively, and the HC2 and the

LC2 of SEQ ID NOs: 10 and 9, respectively.

The invention provides for a pharmaceutical composition comprising the bispecific anti-TNF-α/IL-17A antibody of the invention.

The invention also provides for a method of treating a TNF-α- and/or an IL-17A-mediated inflammatory disease, comprising administering to a subject in need thereof a therapeutically effective amount of the bispecific anti-TNF-α/IL-17A antibody of claim 11 for a time sufficient to treat the TNF-α- and/or the IL-17A-mediated inflammatory disease.

The invention also provides for an anti-idiotypic antibody specifically binding the bispecific anti-TNF-α/IL-17A antibody of the invention.

The invention also provides for a kit comprising the bispecific anti-TNF-α/IL-17A antibody of the invention.

The invention also provides for an isolated synthetic polynucleotide encoding the HC1, the LC1, the HC2 and/or the LC2 of the invention; or comprising a polynucleotide sequence of SEQ ID NOs: 33, 34, 35, 36, 37 or 38.

The invention also provides for a vector comprising the polynucleotide of the invention.

The invention also provides for a host cell comprising the vector of the invention. The invention also provides for a method of producing the isolated bispecific anti-TNF-α/IL-17A antibody of the invention, comprising:
combining an isolated monospecific bivalent anti-TNF-α antibody comprising two heavy chains of SEQ ID NO: 5 or two heavy chains of SEQ ID NO: 7 and two light chains of SEQ ID NO: 6 and an isolated monospecific bivalent anti-IL-17A antibody comprising two heavy chains of SEQ ID NO: 8 or two heavy chains of SEQ ID NO: 10 and two light chains of SEQ ID NO: 9 in a mixture of about 1:1 molar ratio;
introducing a reducing agent into the mixture;
incubating the mixture about ninety minutes to about six hours; removing the reducing agent; and
purifying
   the bispecific anti-TNF-α/IL-17A antibody that comprises a first heavy chain of SEQ ID NO: 5 and a second heavy chain of SEQ ID NO: 8, a first light chain of SEQ ID NO: 6 and a second light chain of SEQ ID NO: 9, wherein the first heavy chain of SEQ ID NO: 5 pairs with the first light chain of SEQ ID NO: 6 to form a first binding domain that specifically binds TNF-α, and the second heavy chain of SEQ ID NO: 8 pairs with the second light chain of SEQ ID NO: 9 to form a second binding domain that specifically binds IL-17A; or purifying the bispecific anti-TNF-α/IL-17A antibody that comprises a first heavy chain of SEQ ID NO: 7 and a second heavy chain of SEQ ID NO: 10, a first light chain of SEQ ID NO: 6 and a second light chain of SEQ ID NO: 9, wherein the first heavy chain of SEQ ID NO: 7 pairs with the first light chain of SEQ ID NO: 6 to form the first binding domain that specifically binds TNF-α, and the second heavy chain of SEQ ID NO: 10 pairs with the second light chain of SEQ ID NO: 9 to form the second binding domain that specifically binds IL-17A.

The invention also provides for an isolated anti-TNF-α antibody comprising the VH of SEQ ID NO: 11 and the VL of SEQ ID NO: 12.

The invention also provides for a pharmaceutical composition comprising the anti-TNF-α antibody of the invention and a pharmaceutically acceptable excipient.

The invention also provides for an isolated synthetic polynucleotide
encoding the VH or the VH and the VL of the anti-TNF-α antibody of the invention; or
comprising a polynucleotide sequence of SEQ ID NO: 39 or SEQ ID NO: 39 and SEQ ID NO: 40.

The invention also provides for a method of treating a TNF-α mediated disease, comprising administering to a subject in need thereof the isolated anti-TNF-α antibody of the invention for a time sufficient to treat the TNF-α mediated disease.

### BRIEF DESCRIPTION OF THE DRAWINGS

FIG. 1A shows IL-6 release from human RA synoviocytes treated with indicated concentrations of IL-17A and TNF-α.

FIG. 1B shows MMP3 release from human RA synoviocytes treated with indicated concentrations of IL-17A and TNF-α.

FIG. 1C shows GROα release from human RA synoviocytes treated with indicated concentrations of IL-17A and TNF-α.

FIG. 2A shows IL-6 release from co-cultures of human primary chondrocytes and RA fibroblast like synoviocytes (FLS) treated with IL-17A, TNF-α or a combination of IL-17A and TNF-α.

FIG. 2B shows neutralization of IL-6 release from co-cultures of human primary chondrocytes and RA fibroblast like synoviocytes (FLS) treated with a combination of IL-17A and TNF-α by mAb 1787 (isotype control mAb), mAb 9809 (anti-TNF-α mAb), mAb 4782 (anti-IL-17A mAb) or mAb 9762 (bispecific anti-TNF-α/IL-17A mAb).

FIG. 3A shows neutralization of IL-17A induced total cell influx into the bronchoalveolar lavage (BAL) in Balb/c mice by isotype control mAb (mAb 1787), anti-IL-17A mAb (mAb 7024) or by bispecific anti-TNF-α/IL-17A mAb (mAb 9762) at indicated doses. Statistical significance was assessed using one way ANOVA with post-hoc Dunnett test. **p<00.1; ***p<0.001. rh: recombinant human; i.n.: intranasal.

FIG. 3B shows neutralization of IL-17A induced neutrophil influx into the bronchoalveolar lavage (BAL) in Balb/c mice by isotype control mAb (mAb 1787), anti-IL-17A mAb (mAb 7024) or by bispecific anti-TNF-α/IL-17A mAb (mAb 9762) at indicated doses. Statistical significance was assessed using one way ANOVA with post-hoc Dunnett test. ***p<0.001. rh: recombinant human; i.n.: intranasal.

FIG. 4A shows total cell influx into the BAL in Balb/c mice in response to intranasal co-treatment of mice with indicated doses of human (hu) TNF-α and/or IL-17A. Data represent mean+SE. Asterisks (*) denote significance. *p<0.05; **p<0.01; ***p<0.001; ****p<0.000 ANOVA followed by post-hoc Dunnett test.

FIG. 4B shows neutrophil influx into the BAL in Balb/c mice in response to intranasal co-treatment of mice with indicated doses of human (hu) TNF-α and/or IL-17A. Data represent mean+SE. Asterisks (*) denote significance. *p<0.05; **p<0.01; ***p<0.001; ****p<0.000 ANOVA followed by post-hoc Dunnett test.

FIG. 5A shows inhibition of total cell influx into the BAL of mice following intranasal instillation of recombinant human TNF-α and IL-17A in combination by the indicated antibodies at indicated doses. mAb 1787: isotype control; mAb 4782: anti-IL-17A antibody; mAb 9809: anti-TNF-α antibody; mAb 9762: bispecific anti-TNF-α/IL-17A antibody. Data represent mean+SE. Data were transformed to log data and statistical analysis performed using one-way ANOVA/Tukey's Multiple Comparison Test. Asterisks (*) denote significance. *p<0.05; **p<0.01; ***p<0.001; ****p<0.000 ANOVA followed by post-hoc test. NS: not significant.

FIG. 5B shows inhibition of neutrophil influx into the BAL of mice following intranasal instillation of recombinant human TNF-α and IL-17A in combination by the indicated antibodies at indicated doses. mAb 1787: isotype control; mAb 4782: anti-IL-17A antibody; mAb 9809: anti-TNF-α antibody; mAb 9762: bispecific anti-TNF-α/IL-17A antibody. Data represent mean+SE. Data were transformed to log data and statistical analysis performed using one-way ANOVA/Tukey's Multiple Comparison Test. Asterisks (*) denote significance. *p<0.05; **p<0.01; ***p<0.001; ****p<0.000 ANOVA followed by post-hoc test. NS: not significant.

### DETAILED DESCRIPTION OF THE INVENTION

All publications, including but not limited to patents and patent applications, cited in this specification are herein incorporated by reference as though fully set forth.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

“Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (KD) of about 1×10⁻⁸ M or less, for example about 1×10⁻⁹ M or less, about 1×10¹⁰ M or less, about 1×10⁻¹¹ M or less, or about 1×10⁻¹² M or less, typically with the KD that is at least one hundred fold less than its KD for binding to a non-specific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human, mouse, rat or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibody molecules” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, (1970) J Exp Med 132:211-50; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, (1987) Mol Biol 196:901-17). The International ImMunoGeneTics (IMGT) database (http://www_imgt_org) provides a standardized numbering and definition of antigen-binding sites. The correspondence between CDRs, HVs and IMGT delineations is described in Lefranc et al., (2003) Dev Comparat Immunol 27:55-77. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated otherwise.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may assigned to one of two clearly distinct types, namely kappa (κ) and lambda (λ), based on the amino acid sequences of their constant domains.

“Antigen-binding fragment” refers to a portion of an immunoglobulin molecule that retains the antigen binding properties of the parental full length antibody. Exemplary antigen-binding fragments are heavy chain complementarity determining regions (HCDR) 1, 2 and/or 3, light chain complementarity determining regions (LCDR) 1, 2 and/or 3, a heavy chain variable region (VH), or a light chain variable region (VL), Fab, F(ab′)2, Fd and Fv fragments as well as domain antibodies (dAb) consisting of either one VH domain or one VL domain. VH and VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody; described for example in Int. Pat. Publ. No. WO1998/44001, Int. Pat. Publ. No. WO1988/01649; Int. Pat. Publ. No. WO1994/13804; Int. Pat. Publ. No. WO1992/01047.

“Monoclonal antibody” refers to an antibody population with single amino acid composition in each heavy and each light chain, except for possible well known alterations such as removal of C-terminal lysine from the antibody heavy chain, and intentionally made asymmetrical substitutions into the heavy chains for example to promoter heterodimer formation when generating bispecific full length antibodies, or to facilitate purification of antibodies using protein A columns Monoclonal antibodies typically bind one antigenic epitope, except that bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Isolated” refers to a homogenous population of molecules (such as synthetic polynucleotides or antibodies) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin. If the antibody contains a constant region or a portion of the constant region, the constant region also is derived from sequences of human origin.

Human antibody comprises heavy or light chain variable regions that are “derived from” sequences of human origin if the variable regions of the antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci as described herein. “Human antibody” may contain amino acid differences when compared to the human germline immunoglobulin or rearranged immunoglobulin genes due to for example naturally occurring somatic mutations or intentional introduction of substitutions into the framework or antigen binding site, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462.

Human antibodies derived from human immunoglobulin sequences may be generated using systems such as phage display incorporating synthetic CDRs and/or synthetic frameworks, or may be subjected to in vitro mutagenesis to improve antibody properties, resulting in antibodies that are not expressed by the human antibody germline repertoire in vivo.

Antibodies in which antigen binding sites are derived from a non-human species are not included in the definition of “human antibody”.

“Recombinant” refers to antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

“Paratope” refers to a portion of an antibody to which an antigen specifically binds. A paratope may be linear in nature or may be discontinuous, formed by a spatial relationship between non-contiguous amino acids of an antibody rather than a linear series of amino acids. A “light chain paratope” and a “heavy chain paratope” or “light chain paratope amino acid residues” and “heavy chain paratope amino acid residues” refer to antibody light chain and heavy chain residues in contact with an antigen, respectively, or in general, “antibody paratope residues” refer to those antibody amino acids that are in contact with antigen.

“Multispecific” refers to an antibody that specifically binds at least two distinct antigens or two distinct epitopes within the antigens, for example three, four or five distinct antigens or epitopes.

“Bispecific” refers to an antibody that specifically binds two distinct antigens or two distinct epitopes within the same antigen. Bispecific antibody may have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset), or may bind an epitope that is shared between two or more distinct antigens.

“Bispecific anti-TNF-α/IL-17A antibody”, “TNF-α/IL-17A antibody”, “anti-TNF-α/IL-17A antibody” or “antibody that specifically binds TNF-α and IL-17A” refers to a molecule comprising at least one domain specifically binding TNF-α and at least one domain specifically binding IL-17A. The domains specifically binding TNF-α and IL-17A are typically VH/VL pairs. The bispecific anti-TNF-α/IL-17A antibody may be monovalent in terms of its binding to either TNF-α or IL-17A.

“Variant” refers to a polypeptide or a polynucleotide that differs from a reference polypeptide or a reference polynucleotide by one or more modifications, for example one or more substitutions, insertions or deletions.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

“Polynucleotide” refers to a molecule comprising a chain of nucleotides covalently linked by a sugar-phosphate backbone or other equivalent covalent chemistry. cDNA is a typical example of a synthetic polynucleotide.

“Polypeptide” or “protein” refers to a molecule that comprises at least two amino acid residues linked by a peptide bond to form a polypeptide Small polypeptides of less than 50 amino acids may be referred to as “peptides”.

“Tumor necrosis factor”, “TNF” or “TNF-α” refers to the well-known human TNF-α. TNF-α is found as a soluble protein as well as a precursor form called transmembrane TNF-α that is expressed as a cell surface type II polypeptide. Transmembrane TNF-α is processed by metalloproteinases such as TNF-α-converting enzyme (TACE) between residues Ala76 and Va177, resulting in the release of the soluble form of TNF-α of 157 amino acid residues. Soluble TNF-α is a homotrimer of 17-kDa cleaved monomers. Transmembrane TNF-α also exists as a homotrimer of 26-kD uncleaved monomers. “TNF-α” encompasses both the soluble and the transmembrane forms. The amino acid sequence of the transmembrane TNF-α is shown in SEQ ID NO: 1. The amino acid sequence of the soluble TNF-α shown in SEQ ID NO: 2.

SEQ ID NO: 1
MSTESMIRDVELAEEALPKKTGGPQGSRRCLFLSLFSFLIVAGATTLFCL
LHFGVIGPQREEFPRDLSLISPLAQAVRSSSRTPSDKPVAHVVANPQAEG
QLQWLNRRANALLANGVELRDNQLVVPSEGLYLIYSQVLFKGQGCPSTHV
LLTHTISRIAVSYQTKVNLLSAIKSPCQRETPEGAEAKPWYEPIYLGGVF
QLEKGDRLSAEINRPDYLDFAESGQVYFGHAL
SEQ ID NO: 2
VRSSSRTPSDKPVAHVVANPQAEGQLQWLNRRANALLANGVELRDNQLVV
PSEGLYLIYSQVLFKGQGCPSTHVLLTHTISRIAVSYQTKVNLLSAIKSP
CQRETPEGAEAKPWYEPIYLGGVFQLEKGDRLSAEINRPDYLDFAESGQV
YFGHAL

“IL-17A” or “interleukin-17A” refers to human IL-17A. The amino acid sequence of the mature human IL-17A is shown in SEQ ID NO: 3. IL-17A exists in vivo as a homodimer or a heterodimer in complex with IL-17F (known as “IL-17A/F”). “IL-17A” encompasses the IL-17A monomer, the IL-17A homodimer and the IL-17A/F heterodimer. The amino acid sequence of the mature IL-17F is shown in SEQ ID NO: 4.

SEQ ID NO: 3
GITIPRNPGCPNSEDKNFPRTVMVNLNIHNRNTNTNPKRSSDYYNRSTSP
WNLHRNEDPERYPSVIWEAKCRHLGCINADGNVDYHMNSVPIQQEILVLR
REPPHCPNSFRLEKILVSVGCTCVTPIVHHVA
SEQ ID NO: 4
RKIPKVGHTFFQKPESCPPVPGGSMKLDIGIINENQRVSMSRNIESRSTS
PWNYTVTWDPNRYPSEVVQAQCRNLGCINAQGKEDISMNSVPIQQETLVV
RRKHQGCSVSFQLEKVLVTVGCTCVTPVIHHVQ

“In combination with” means that two or more therapeutics are administered to a subject together in a mixture, concurrently as single agents or sequentially as single agents in any order.

“Sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, liquid solutions contacted with a subject or biological source, for example, cell and organ culture medium including cell or organ conditioned medium, lavage fluids and the like, tissue biopsies, fine needle aspirations or surgically resected tumor tissue, or synovial biopsies.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist may be an antibody, a soluble ligand, a small molecule, a DNA or RNA such as siRNA. An exemplary antagonist is an antagonistic bispecific anti-TNF-α/IL-17A antibody. A typical reaction or activity that is induced by TNF-α binding to its receptor TNFαR1 or TNFαR2 is TRAF2-mediated activation of nuclear factor-κB (NFκB) pathway, activation of MAP3K (ASK-1), which in turn activates c-Jun N-terminal kinases (JNKs) and p38 MAPK, or activation of MEK-ERK pathway, resulting in activation of transcription of many downstream genes and amplification of inflammatory responses including induction of cytokine production, activation and expression of adhesion molecules, and growth stimulation. A typical reaction or activity that is induced by IL-17A binding to its receptor IL-17RA/IL-17RC is TRAF6-mediated activation of nuclear factor-κB (NF-κB) pathway, increased expression of granulocyte colony-stimulating factor (G-CSF) as well as chemokine ligands for CXCR2, including chemokine CXC motif ligand 1 (CXCL1), CXCL2 and CXCL8, recruitment and activation of neutrophils, lymphocytes and macrophages leading to local inflammation and tissue damage. Assays measuring the typical reactions or activity induced by TNF-α and IL-17A are known and described herein.

“Subject” or “patient” as used interchangeably includes any human or nonhuman animal “Nonhuman animal” includes all vertebrates, e.g., mammals and non-mammals, such as nonhuman primates, sheep, dogs, cats, horses, cows chickens, amphibians, reptiles, etc.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated.

Conventional one and three-letter amino acid codes are used herein as shown in Table 1.

TABLE 1

| | Three- | One- |
|---|---|---|
| Amino acid | letter code | letter code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Compositions of Matter

The present invention provides bispecific anti-TNF-α/IL-17A antibodies that simultaneously antagonize both TNF-α and IL-17A, polynucleotides encoding the antibodies, vectors, host cells, and methods of using the antibodies.

The invention also provides for an isolated bispecific anti-tumor necrosis factor (TNF-α)/interleukin-17A (IL-17A) antibody comprising a first domain specifically binding TNF-α and a second domain specifically binding IL-17A.

The invention also provides for an isolated bispecific anti-TNF-α/IL-17A antibody comprising a first domain specifically binding TNF-α and a second domain specifically binding IL-17A, wherein the first domain comprises a heavy chain complementarity determining region (HCDR) 1, a HCDR2, a HCDR3, a light chain complementarity determining region (LCDR) 1, a LCDR2 and a LCDR3 of SEQ ID NOs: 15, 16, 17, 18, 19 and 20, respectively, and the second domain comprises the HCDR1, the HCDR2, the HCDR3, the LCDR1, the LCDR2 and the LCDR3 of SEQ ID NOs: 21, 22, 23, 24, 25 and 26, respectively.

In some embodiments, the bispecific anti-TNF-α/IL-17A antibody binds TNF-α with an equilibrium dissociation constant (K_{D}) of less than about 3×10⁻¹¹ M, when the K_{D} is measured using Biacore 3000 system at 25° C. in PBS containing 0.01% polysorbate 20 (PS-20) and 100 μg/ml bovine serum albumin.

In some embodiments, the bispecific anti-TNF-α/IL-17A antibody binds IL-17A with an equilibrium dissociation constant (K_{D}) of less than about 5×10⁻¹¹ M, when the K_{D} is measured using Biacore 3000 system at 25° C. in PBS containing 0.01% polysorbate 20 (PS-20) and 100 μg/ml bovine serum albumin.

In some embodiments, the bispecific anti-TNF-α/IL-17A antibody binds TNF-α and IL-17A with an equilibrium dissociation constant (K_{D}) of less than about 3×10⁻¹¹ M and less than about 5×10⁻¹¹ M, respectively, when the K_{D} is measured using Biacore 3000 system at 25° C. in PBS containing 0.01% polysorbate 20 (PS-20) and 100 μg/ml bovine serum albumin.

Exemplary such bispecific anti-TNF-α/IL-17A antibodies are mAb 9762 and mAb 8759 described herein.

The affinity of an antibody to TNF-α or IL-17A may be determined experimentally using any suitable method. Such methods may utilize ProteOn XPR36, Biacore 3000 or KinExA instrumentation, ELISA or competitive binding assays known to those skilled in the art. The measured affinity of a particular antibody/antigen interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer, such as the buffer described herein. Skilled in the art will appreciate that the internal error for affinity measurements for example using Biacore 3000 or ProteOn (measured as standard deviation, SD) may typically be within 5-33% for measurements within the typical limits of detection. Therefore the term “about” in the context of K_{D} reflects the typical standard deviation in the assay. For example, the typical SD for a K_{D} of 1×10⁻⁹M is up to +0.33×10⁻⁹M.

The bispecific anti-TNF-α/IL-17A antibodies of the invention described herein are antagonists of TNF-α and IL-17A.

The bispecific anti-TNF-α/IL-17A antibodies of the invention may be tested for their antagonistic activity using assays described herein. An exemplary assay is an assay to evaluate inhibition of recombinant human TNF-α-mediated cytotoxicity of cells expressing TNF-α receptors such as WEHI-164 mouse fibrosarcoma cells or KYM-1D4 human rhabdomyosarcoma cells. Another exemplary assay is an assay to evaluate inhibition of IL-17A-mediated IL-6 or GROα production from human dermal fibroblasts. Exemplary assays that may be used are described herein in the Examples.

In some embodiments, the bispecific anti-TNF-α/IL-17A antibody inhibits IL-17A/F-mediated IL-6 production by normal human dermal fibroblasts with an IC₅₀ value of between about 0.05 μg/ml and about 0.3 μg/ml and recombinant human TNF-α-mediated cytotoxicity in KYM-1D4 human rhabdomyosarcoma cell line cells with an IC₅₀ value of between about 0.02 nM and about 0.2 nM.

In some embodiments, the first domain comprises a heavy chain variable region (VH) and a light chain variable region (VL) of SEQ ID NOs: 11 and 12, respectively.

In some embodiments, the first domain comprises a heavy chain variable region (VH) and a light chain variable region (VL) of SEQ ID NOs: 11 and 12, respectively and the second domain comprises the VH and the VL of SEQ ID NOs: 13 and 14, respectively.

In some embodiments, the bispecific isolated bispecific anti-TNF-α/IL-17A antibody is an IgG1 isotype.

In some embodiments, the bispecific isolated bispecific anti-TNF-α/IL-17A antibody is an IgG2 isotype.

In some embodiments, the bispecific isolated bispecific anti-TNF-α/IL-17A antibody is an IgG3 isotype.

In some embodiments, the bispecific isolated bispecific anti-TNF-α/IL-17A antibody is an IgG4 isotype.

In some embodiments, the bispecific anti-TNF-α/IL-17A antibody comprises an arginine (R) at position 409 in a first antibody heavy chain (HC1) and a leucine (L) at position 405 in a second antibody heavy chain (HC2), wherein residue numbering is according to the EU Index.

In some embodiments, the bispecific anti-TNF-α/IL-17A antibody optionally comprises M252Y, S254T and T256E substitutions in the HC1, the HC2 or the HC1 and the HC2, wherein residue numbering is according to the EU Index.

In some embodiments, the bispecific anti-TNF-α/IL-17A antibody is humanized or human.

In some embodiments, the bispecific anti-TNF-α/IL-17A antibody comprises a first heavy chain (HC1) and a first light chain (LC1) of SEQ ID NOs: 5 and 6, respectively, and a second heavy chain (HC2) and a second light chain (LC2) of SEQ ID NOs: 8 and 9, respectively.

In some embodiments, the bispecific anti-TNF-α/IL-17A antibody comprises THE HC1 and the LC1 of SEQ ID NOs: 7 and 6, respectively, and the HC2 and the LC2 of SEQ ID NOs: 10 and 9, respectively.

The invention also provides for an isolated bispecific anti-TNF-α/IL-17A antibody comprising a first domain specifically binding TNF-α, a second domain specifically binding IL-17A, an arginine (R) at position 409 in a first antibody heavy chain (HC1) and a leucine (L) at position 405 in a second antibody heavy chain (HC2), wherein amino acid residue numbering is according to the EU Index, wherein
the first domain comprises a heavy chain complementarity determining region (HCDR) 1, a HCDR2 and a HCDR3 contained in a heavy chain variable region (VH) having an amino acid sequence of SEQ ID NO: 11 and a light chain complementarity determining region (LCDR) 1, a LCDR2 and a LCDR3 contained in a light chain variable region (VL) having an amino acid sequence of SEQ ID NO: 12, and
the second domain comprises the HCDR1, the HCDR2 and the HCDR3 contained in the VH having an amino acid sequence of SEQ ID NO: 13 and the LCDR1, the LCDR2 and the LCDR3 contained in the VL having an amino acid sequence of SEQ ID NO: 14, wherein the HCDR1, the HCDR2, the HCDR3, the LCDR1, the LCDR2 and the LCDR3 are defined according to Kabat, Chothia or IMGT.

The invention also provides for an isolated bispecific anti-TNF-α/IL-17A antibody comprising a first domain specifically binding TNF-α, a second domain specifically binding IL-17A, an arginine (R) at position 409 in a first antibody heavy chain (HC1) and a leucine (L) at position 405 in a second antibody heavy chain (HC2), wherein amino acid residue numbering is according to the EU Index, wherein the first domain comprises the HCDR1, the HCDR2, the HCDR3, the LCDR1, the LCDR2 and the LCDR3 of SEQ ID NOs: 15, 16, 17, 18, 19 and 20, respectively, and the second domain comprises the HCDR1, the HCDR2, the HCDR3, the LCDR1, the LCDR2 and the LCDR3 of SEQ ID NOs: 21, 22, 23, 24, 25 and 26, respectively.

The invention also provides for an isolated bispecific anti-TNF-α/IL-17A antibody comprising a first domain specifically binding TNF-α, a second domain specifically binding IL-17A, an arginine (R) at position 409 in a first antibody heavy chain (HC1) and a leucine (L) at position 405 in a second antibody heavy chain (HC2), wherein the first domain comprises the VH and the VL of SEQ ID NOs: 11 and 12, respectively, and amino acid residue numbering is according to the EU Index.

The invention also provides for an isolated bispecific anti-TNF-α/IL-17A antibody comprising a first domain specifically binding TNF-α and a second domain specifically binding IL-17A, comprising a first heavy chain and a first light chain of SEQ ID NOs: 5 and 6, respectively, and a second heavy chain and a second light chain of SEQ ID NOs: 8 and 9, respectively.

The invention also provides for an isolated bispecific anti-TNF-α/IL-17A antibody comprising a first domain specifically binding TNF-α and a second domain specifically binding IL-17A, comprising a first heavy chain and a first light chain of SEQ ID NOs: 7 and 6, respectively, and a second heavy chain and a second light chain of SEQ ID NOs: 10 and 9, respectively.

The invention also provides for an isolated anti-TNF-α antibody comprising the VH of SEQ ID NO: 11 and the VL of SEQ ID NO: 12.

### Homologous Antibodies

Variants of the isolated bispecific anti-TNF-α/IL-17A antibodies of the invention are within the scope of the invention. For example, variants may comprise one, two, three, four, five, six, seven, eight, nine, ten, eleven, twelve, thirteen, fourteen or fifteen amino acid substitutions in the heavy or light chain of the antibody as long as the homologous antibodies retain or have improved functional properties when compared to the parental antibodies. In some embodiments, the sequence identity may be about 90%, 91%, 92%, 93%, 94%, 95%, 96%, 97%, 98% or 99% to the heavy chain or the light chain amino acid sequence of the invention.

The invention also provides for an isolated bispecific anti-TNF-α/IL-17A antibody comprising a first domain specifically binding TNF-α and a second domain specifically binding IL-17A, comprising a first heavy chain and a first light chain of SEQ ID NOs: 5 and 6, respectively, and a second heavy chain and a second light chain of SEQ ID NOs: 8 and 9, respectively, wherein the first heavy chain, the first light chain, the second heavy chain and the second light chain optionally comprise one, two, three, four, five, six, seven, eight, nine, ten, eleven, twelve, thirteen, fourteen or fifteen amino acid substitutions. Optionally, any substitutions are not within the CDRs.

The invention also provides for an isolated bispecific anti-TNF-α/IL-17A antibody comprising a first domain specifically binding TNF-α and a second domain specifically binding IL-17A, comprising a first heavy chain and a first light chain of SEQ ID NOs: 7 and 6, respectively, and a second heavy chain and a second light chain of SEQ ID NOs: 10 and 9, respectively, wherein the first heavy chain, the first light chain, the second heavy chain and the second light chain optionally comprise one, two, three, four, five, six, seven, eight, nine, ten, eleven, twelve, thirteen, fourteen or fifteen amino acid substitutions. Optionally, any substitutions are not within the CDRs.

The substitutions in the bispecific anti-TNF-α/IL-17A antibodies of the invention may be conservative modifications. “Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24) Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

### Engineered and Modified Antibodies

The antibodies of the invention may further be engineered to generate modified antibodies with similar or altered properties when compared to the parental antibodies. The VH, the VL, the VH and the VL, the constant regions, VH framework, VL framework, or any or all of the six CDRs may be engineered in the antibodies of the invention.

The CDR residues of the antibodies of the invention may be mutated to improve affinity of the antibodies to TNF-α, IL-17A, or TNF-α and IL-17A.

The CDR residues of the antibodies of the invention may be mutated for example to minimize risk of post-translational modifications Amino acid residues of putative motifs for deamination (NS), acid-catalyzed hydrolysis (DP), isomerization (DS), or oxidation (W) may be substituted with any of the naturally occurring amino acids to mutagenize the motifs, and the resulting antibodies may be tested for their functionality and stability using methods described herein.

Antibodies of the invention may be modified to improve stability, selectivity, cross-reactivity, affinity, immunogenicity or other desirable biological or biophysical property are within the scope of the invention. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn et al., (2001) J Mol Biol 305:989-1010). Potential structure destabilizing residues may be identified based upon the crystal structure of the antibody or by molecular modeling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC).

In general, the protein Tₘ is correlated with its stability and inversely correlated with its susceptibility to unfolding and denaturation in solution and the degradation processes that depend on the tendency of the protein to unfold (Remmele et al., (2000) Biopharm 13:36-46). A number of studies have found correlation between the ranking of the physical stability of formulations measured as thermal stability by DSC and physical stability measured by other methods (Gupta et al., (2003) AAPS PharmSci 5E8; Zhang et al., (2004) J Pharm Sci 93:3076-89; Maa et al., (1996) Int J Pharm 140:155-68; Bedu-Addo et al., (2004) Pharm Res 21:1353-61; Remmele et al., (1997) Pharm Res 15:200-8). Formulation studies suggest that a Fab Tₘ has implication for long-term physical stability of a corresponding mAb.

C-terminal lysine (CTL) may be removed from injected antibodies by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA-Fe³⁺ as described in U.S. Patent Publ. No. US20140273092. CTL content in antibodies can be measured using known methods.

The invention also provides for an isolated bispecific anti-TNF-α/IL-17A antibody having a C-terminal lysine content of about 10% to about 90%, about 20% to about 80%, about 40% to about 70%, about 55% to about 70%, or about 60%.

Fc substitutions may be made to the isolated bispecific anti-TNF-α/IL-17A antibodies of the invention to modulate antibody effector functions and pharmacokinetic properties. In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All of these interactions are initiated through the binding of the Fc domain of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are “activating Fcγ receptors” (i e, immune system enhancing); FcγRIIB (CD32B) is an inhibiting Fcγ receptor” (i.e., immune system dampening). Binding to the FcRn receptor modulates antibody half-life.

The invention also provides for an isolated bispecific anti-TNF-α/IL-17A antibody comprising at least one substitution in an antibody Fc.

The invention also provides for an isolated bispecific anti-TNF-α/IL-17A antibody comprising one, two, three, four, five, six, seven, eight, nine, ten, eleven, twelve, thirteen, fourteen or fifteen substitutions in the antibody Fc.

Fc positions that may be substituted to modulate antibody half-life are those described for example in Dall'Acqua et al., (2006) J Biol Chem 281:23514-240, Zalevsky et al., (2010) Nat Biotechnol 28:157-159, Hinton et al., (2004) J Biol Chem 279(8):6213-6216, Hinton et al., (2006) J Immunol 176:346-356, Shields et al. (2001) J Biol Chem 276:6591-6607, Petkova et al., (2006). Int Immunol 18:1759-1769, Datta-Mannan et al., (2007) Drug Metab Dispos, 35:86-94, 2007, Vaccaro et al., (2005) Nat Biotechnol 23:1283-1288, Yeung et al., (2010) Cancer Res, 70:3269-3277 and Kim et al., (1999) Eur J Immunol 29: 2819, and include positions 250, 252, 253, 254, 256, 257, 307, 376, 380, 428, 434 and 435. Exemplary substitutions that may be made singularly or in combination are substitutions T250Q, M252Y, I253A, S254T, T256E, P2571, T307A, D376V, E380A, M428L, H433K, N434S, N434A, N434H, N434F, H435A and H435R. Exemplary singular or combination substitutions that may be made to increase the half-life of the antibody are substitutions M428L/N434S, M252Y/S254T/T256E, T250Q/M428L, N434A and T307A/E380A/N434A. Exemplary singular or combination substitutions that may be made to reduce the half-life of the antibody are substitutions H435A, P2571/N434H, D376V/N434H, M252Y/S254T/T256E/H433K/N434F, T308P/N434A and H435R.

The invention also provides for an isolated bispecific anti-TNF-α/IL-17A antibody comprising at least one substitution in the antibody Fc at amino acid position 250, 252, 253, 254, 256, 257, 307, 376, 380, 428, 434 or 435.

The invention also provides for an isolated bispecific anti-TNF-α/IL-17A antibody comprising at least one substitution in the antibody Fc selected from the group consisting of T250Q, M252Y, I253A, S254T, T256E, P2571, T307A, D376V, E380A, M428L, H433K, N434S, N434A, N434H, N434F, H435A and H435R.

The invention also provides for an isolated bispecific anti-TNF-α/IL-17A antibody comprising at least one substitution in the antibody Fc selected from the group consisting of M428L/N434S, M252Y/S254T/T256E, T250Q/M428L, N434A, T307A/E380A/N434A, H435A, P2571/N434H, D376V/N434H, M252Y/S254T/T256E/H433K/N434F, T308P/N434A and H435R.

The invention also provides for an isolated bispecific anti-TNF-α/IL-17A antibody comprising at least one substitution in the antibody Fc that reduces binding of the antibody to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be substituted to reduce binding of the antibody to the activating FcγR and subsequently to reduce effector function are those described for example in Shields et al., (2001) J Biol Chem 276:6591-6604, Intl. Patent Publ. No. WO2011/066501, U.S. Pat. Nos. 6,737,056 and 5,624,821, Xu et al., (2000) Cell Immunol, 200:16-26, Alegre et al., (1994) Transplantation 57:1537-1543, Bolt et al., (1993) Eur J Immunol 23:403-411, Cole et al., (1999) Transplantation, 68:563-571, Rother et al., (2007) Nat Biotechnol 25:1256-1264, Ghevaert et al., (2008) J Clin Invest 118:2929-2938, An et al., (2009) mAbs, 1:572-579) and include positions 214, 233, 234, 235, 236, 237, 238, 265, 267, 268, 270, 295, 297, 309, 327, 328, 329, 330, 331 and 365. Exemplary substitutions that may be made singularly or in combination are substitutions K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination substitutions that result in antibodies with reduced ADCC are substitutions L234A/L235A on IgG1, V234A,/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358Mon IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

Well-known S228P substitution may be made in IgG4 antibodies to enhance IgG4 stability.

The invention also provides for an isolated bispecific anti-TNF-α/IL-17A antibody comprising a substitution in at least one residue position 214, 233, 234, 235, 236, 237, 238, 265, 267, 268, 270, 295, 297, 309, 327, 328, 329, 330, 331 or 365, wherein residue numbering is according to the EU Index.

The invention also provides for an isolated bispecific anti-TNF-α/IL-17A antibody comprising at least one substitution selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S, wherein residue numbering is according to the EU Index.

The invention also provides for an isolated bispecific anti-TNF-α/IL-17A antibody comprising a substitution in at least one residue position 228, 234, 235, 237, 238, 268, 330 or 331, wherein residue numbering is according to the EU Index.

The invention also provides for an isolated bispecific anti-TNF-α/IL-17A antibody comprising a S228P substitution, wherein residue numbering is according to the EU Index.

The invention also provides for an isolated bispecific anti-TNF-α/IL-17A antibody comprising a V234A substitution, wherein residue numbering is according to the EU Index.

The invention also provides for an isolated bispecific anti-TNF-α/IL-17A antibody comprising a F234A substitution, wherein residue numbering is according to the EU Index.

The invention also provides for an isolated bispecific anti-TNF-α/IL-17A antibody comprising a G237A substitution, wherein residue numbering is according to the EU Index.

The invention also provides for an isolated bispecific anti-TNF-α/IL-17A antibody comprising a P238S substitution, wherein residue numbering is according to the EU Index.

The invention also provides for an isolated bispecific anti-TNF-α/IL-17A antibody comprising a H268A substitution, wherein residue numbering is according to the EU Index.

The invention also provides for an isolated bispecific anti-TNF-α/IL-17A antibody comprising a Q268A substitution, wherein residue numbering is according to the EU Index.

The invention also provides for an isolated bispecific anti-TNF-α/IL-17A antibody comprising an A330S substitution, wherein residue numbering is according to the EU Index.

The invention also provides for an isolated bispecific anti-TNF-α/IL-17A antibody comprising a P331S substitution, wherein residue numbering is according to the EU Index.

The invention also provides for an isolated bispecific anti-TNF-α/IL-17A antibody comprising L234A, L235A, G237A, P238S, H268A, A330S and P331S substitutions, wherein residue numbering is according to the EU Index.

The invention also provides for an isolated bispecific anti-TNF-α/IL-17A antibody comprising V234A, G237A, P238S, H268A, V309L, A330S and P331S substitutions, wherein residue numbering is according to the EU Index.

The invention also provides for an isolated bispecific anti-TNF-α/IL-17A antibody comprising F234A, L235A, G237A, P238S and Q268A substitutions, wherein residue numbering is according to the EU Index.

The invention also provides for an isolated bispecific anti-TNF-α/IL-17A antibody comprising L234A, L235A or L234A and L235A substitutions, wherein residue numbering is according to the EU Index.

The invention also provides for an isolated bispecific anti-TNF-α/IL-17A antibody comprising F234A, L235A or F234A and L235A substitutions, wherein residue numbering is according to the EU Index.

The invention also provides for an isolated bispecific anti-TNF-α/IL-17A antibody comprising S228P, F234A and L235A substitutions, wherein residue numbering is according to the EU Index.

The invention also provides for an isolated bispecific anti-TNF-α/IL-17A antibody comprising a S228P substitution, wherein residue numbering is according to the EU Index.

### Methods of Generating Homologous Antibodies, Antibodies With Conservative Modifications, and Engineered and Modified Antibodies

The antibodies of the invention that have altered amino acid sequences when compared to the parental antibodies may be generated using standard cloning and expression technologies. For example, site-directed mutagenesis or PCR-mediated mutagenesis may be performed to introduce the mutation(s) and the effect on antibody binding or other property of interest, may be evaluated using well known methods and the methods described herein in the Examples.

### Antibody Allotypes

The antibodies of the invention may be an IgG1, IgG2, IgG3 or IgG4 isotype.

In some embodiments, the bispecific anti-TNF-α/IL-17A antibody of the invention is an IgG1, an IgG2, an IgG3 or an IgG4 isotype.

Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host may be determined in part by the allotype of the antibody (Stickler et al., (2011) Genes and Immunity 12:213-21). Antibody allotype is related to amino acid sequence variations at specific locations in the constant region sequences of the antibody. Table 2 shows select IgG1, IgG2 and IgG4 allotypes.

The invention also provides for an isolated bispecific anti-TNF-α/IL-17A antibody having a G2m(n) allotype.

The invention also provides for an isolated bispecific anti-TNF-α/IL-17A antibody having a G2m(n-) allotype.

The invention also provides for an isolated bispecific anti-TNF-α/IL-17A antibody having a G2m(n)/(n-) allotype.

The invention also provides for an isolated bispecific anti-TNF-α/IL-17A antibody having a G4m(a) allotype.

The invention also provides for an isolated bispecific anti-TNF-α/IL-17A antibody having a G1m(17) allotype.

The invention also provides for an isolated bispecific anti-TNF-α/IL-17A antibody having a G1m(17,1) allotype.

TABLE 2

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 409 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| G1m(17) | | | | | K | E | M | A |
| G1m(17, 1) | | | | | K | D | L | A |

### Anti-Idiotypic Antibodies

The invention also provides for an anti-idiotypic antibody specifically binding to the bispecific anti-TNF-α/IL-17A antibody of the invention.

The invention also provides for an anti-idiotypic antibody specifically binding the antibody comprising the HC1, the LC1, the HC2 and the LC2 of SEQ ID NOs: 5, 6, 8 and 9, respectively.

The invention also provides for an anti-idiotypic antibody specifically binding the antibody comprising the HC1, the LC1, the HC2 and the LC2 of SEQ ID NOs: 7, 6, 10 and 9, respectively.

An anti-idiotypic (Id) antibody is an antibody which recognizes the antigenic determinants (e.g. the paratope or CDRs) of the antibody. The Id antibody may be antigen-blocking or non-blocking. The antigen-blocking Id may be used to detect the free antibody in a sample (e.g. bispecific anti-TNF-α/IL-17A antibody of the invention). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original mAb, which induced the anti-Id. Thus, by using antibodies to the idiotypic determinants of a mAb, it is possible to identify other clones expressing antibodies of identical specificity. Anti-Id antibodies may be varied (thereby producing anti-Id antibody variants) and/or derivatized by any suitable technique, such as those described elsewhere herein with respect to the antibodies specifically binding to the bispecific anti-TNF-α/IL-17A antibody of the invention.

### Generation of the Bispecific Anti-TNF-α/IL-17A Antibodies of the Invention

The bispecific anti-TNF-α/IL-17A antibodies of the invention may be generated by combining TNF-α binding VH/VL domains with IL-17A binding VH/VL domains isolated de novo or by using VH/VL domains from publicly available monospecific anti-TNF-α and anti-IL-17A antibodies, and/or by mix-matching the TNF-α or IL-17A binding VH/VL domains identified herein with publicly available TNF-α or IL-17A binding VH/VL domains

Anti-TNF-α and anti-IL-17A antibodies to be used to generate the bispecific antibodies of the invention may be generated de novo using various technologies. For example, the hybridoma method of Kohler and Milstein, Nature 256:495, 1975 may be used to generate monoclonal antibodies. In the hybridoma method, a mouse or other host animal, such as a hamster, rat or monkey, is immunized with human or cyno TNF-α or IL-17A antigens, followed by fusion of spleen cells from immunized animals with myeloma cells using standard methods to form hybridoma cells (Goding, Monoclonal Antibodies: Principles and Practice, pp.59-103 (Academic Press, 1986)). Colonies arising from single immortalized hybridoma cells are screened for production of antibodies with desired properties, such as specificity of binding, cross-reactivity or lack thereof, and affinity for the antigen.

Various host animals may be used to produce the anti-TNF-α or anti-IL-17A antibodies to be used to generate the bispecific antibodies of the invention described herein. For example, Balb/c mice may be used to generate mouse anti-human TNF-α or IL-17A antibodies. The antibodies made in Balb/c mice and other non-human animals may be humanized using various technologies to generate more human-like sequences.

Exemplary humanization techniques including selection of human acceptor frameworks are known and include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709, 226). In these methods, CDRs of parental antibodies are transferred onto human frameworks that may be selected based on their overall homology to the parental frameworks, based on similarity in CDR length, or canonical structure identity, or a combination thereof.

Humanized antibodies may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs.

Transgenic animals, such as mice or rat carrying human immunoglobulin (Ig) loci in their genome may be used to generate human antibodies against TNF-α or IL-17A, and are described in for example U.S. Pat. No. 6,150,584, Int. Patent Publ. No. WO99/45962, Int. Patent Publ. Nos. WO2002/066630, WO2002/43478, WO2002/043478 and WO1990/04036, Lonberg et al (1994) Nature 368:856-9; Green et al (1994) Nature Genet. 7:13-21; Green & Jakobovits (1998) Exp. Med. 188:483-95; Lonberg and Huszar (1995) Int Rev Immunol 13:65-93; Bruggemann et al., (1991) Eur J Immunol 21:1323-1326; Fishwild et al., (1996) Nat Biotechnol 14:845-851; Mendez et al., (1997) Nat Genet 15:146-156; Green (1999) J Immunol Methods 231:11-23; Yang et al., (1999) Cancer Res 59:1236-1243; Brüggemann and Taussig (1997) Curr Opin Biotechnol 8:455-458. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtincnet), KyMab (http://_www_kymab_com), Trianni (http://_www.trianni_com) and Ablexis (http://_www_ablexis_com) may be engaged to provide human antibodies directed against a selected antigen using technologies as described above.

Human antibodies may be selected from a phage display library, where the phage is engineered to express human immunoglobulins or portions thereof such as Fabs, single chain antibodies (scFv), or unpaired or paired antibody variable regions (Knappik et al., (2000) J Mol Biol 296:57-86; Krebs et al., (2001) J Immunol Meth 254:67-84; Vaughan et al., (1996) Nature Biotechnology 14:309-314; Sheets et al., (1998) PITAS (USA) 95:6157-6162; Hoogenboom and Winter (1991) J Mol Biol 227:381; Marks et al., (1991) J Mol Biol 222:581). The antibodies binding TNF-α or IL-17A to be used to generate the bispecific anti-TNF-α/IL-17A antibodies of the invention may be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries may be screened for phage binding to human and/or cyno TNF-α or IL-17A and the obtained positive clones may be further characterized, the Fabs isolated from the clone lysates, and expressed as full length IgGs. Such phage display methods for isolating human antibodies are described in for example U.S. Pat. Nos. 5,223,409, 5,403,484, 5,571,698, 5,427,908, 5, 580,717, 5,969,108, 6,172,197, 5,885,793; 6,521,404; 6,544,731; 6,555,313; 6,582,915 and 6,593,081.

Preparation of immunogenic antigens and monoclonal antibody production may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

Bispecific antibodies of the invention comprise antibodies having a full length antibody structure.

“Full length antibody” refers to an antibody having two full length antibody heavy chains and two full length antibody light chains A full length antibody heavy chain (HC) consists of well-known heavy chain variable and constant domains VH, CH1, hinge, CH2, and CH3. A full length antibody light chain (LC) consists of well-known light chain variable and constant domains VL and CL. The full length antibody may be lacking the C-terminal lysine (K) in either one or both heavy chains.

“Fab-arm” or “half molecule” refers to one heavy chain-light chain pair that specifically binds an antigen.

Full length bispecific antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on TNF-α and an epitope on IL-17A.

“Homodimerization” refers to an interaction of two heavy chains having identical CH3 amino acid sequences. “Homodimer” refers to an antibody having two heavy chains with identical CH3 amino acid sequences.

“Heterodimerization” refers to an interaction of two heavy chains having non-identical CH3 amino acid sequences. “Heterodimer” refers to an antibody having two heavy chains with non-identical CH3 amino acid sequences.

The bispecific antibodies include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody)(EMD Serono), the Biclonic (Merus) and the DuoBody (Genmab A/S).

The Triomab quadroma technology may be used to generate full length bispecific antibodies of the invention. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., Intl. Publ. No. WO 2006/028936) may be used to generate full length bispecific antibodies of the invention. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology may be used to generate full length bispecific antibodies of the invention. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351YF405A_Y407V/T350V_T366L_K392L_T394W as described in U.S. Patent Publ. No. US2012/0149876 or U.S. Patent Publ. No. US2013/0195849.

LUZ-Y technology may be utilized to generate bispecific antibodies of the invention. In this technology, a leucine zipper is added into the C terminus of the CH3 domains to drive the heterodimer assembly from parental mAbs that is removed post-purification as described in Wranik et al., (2012) J Biol Chem 287(52): 42221-9.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

The bispecific anti-TNF-α/IL-17A antibodies of the invention may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Int. Patent Publ. No. WO2011/131746 (DuoBody technology). In the methods, the first monospecific bivalent antibody (e.g., anti-TNF-α antibody) and the second monospecific bivalent antibody (e.g., anti-IL-17A antibody) are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

The invention also provides for an isolated bispecific anti-TNF-α/IL-17A antibody comprising a first domain specifically binding TNF-α, a second domain specifically binding IL-17A, and at least one substitution in an antibody CH3 constant domain.

In some embodiments, the at least one substitution in the antibody CH3 constant domain is K409R, F405L or F405L and R409K substitution, wherein residue numbering is according to the EU Index.

Antibody domains and numbering are well known. “Asymmetrical” refers to non-identical substitutions in the two CH3 domains in two separate heavy chains in an antibody. An IgG1 CH3 region typically consists of residues 341-446 on IgG1 (residue numbering according to the EU index).

The invention also provides for an isolated bispecific anti-TNF-α/IL-17A antibody comprising a first domain specifically binding TNF-α, a second domain specifically binding IL-17A and a F405L substitution in an antibody first heavy chain (HC1) and a K409R substitution in an antibody second heavy chain (HC2).

In some embodiments described herein, the isolated bispecific anti-TNF-α/IL-17A antibody comprises V234A, G237A, P238S, H268A, V309L, A330S, P331S and K409R substitutions in the HC1 and V234A, G237A, P238S, H268A, V309L, A330S, P331S and F405L substitutions in the HC2, wherein the antibody is of IgG2 isotype.

In some embodiments described herein, the isolated bispecific anti-TNF-α/IL-17A antibody comprises a S228P substitution in the HC1 and S228P, F405L and R409K substitutions in the HC2, wherein the antibody is an IgG4 isotype.

In some embodiments described herein, the bispecific antibody of the invention comprises at least one, two, three, four, five, six, seven or eight asymmetrical substitutions in the HC1 and the HC2 at residue positions 350, 366, 368, 370, 399, 405, 407 or 409, when residue numbering is according to the EU index.

In some embodiments described herein, the bispecific antibody of the invention comprises at least one, two, three or four asymmetrical substitutions in the HC1 and the HC2 at residue positions 350, 370, 405 or 409, when residue numbering is according to the EU index.

In some embodiments described herein, the HC1 comprises a K409R substitution or a F405L substitution and the HC2 comprises a K409R substitution or a F405L substitution, wherein residue numbering is according to the EU index.

In some embodiments described herein, the HC1 comprises the F405L substitution and the HC2 comprises the K409R substitution.

Substitutions are typically made at the DNA level to a molecule such as the constant domain of the antibody using standard methods.

The antibodies of the invention may be engineered into various well known antibody forms.

In some embodiments, the bispecific antibody of the present invention is a diabody or a cross-body.

In some embodiments, the bispecific antibodies include recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, recombinant IgG-like dual targeting molecules include Dual Targeting (DT)-Ig (GSK/Domantis), Two-in-one Antibody (Genentech) and mAb2 (F-Star).

In some embodiments, IgG fusion molecules include Dual Variable Domain (DVD)-Ig (Abbott), Ts2Ab (Medlmmune/AZ) and BsAb (Zymogenetics), HERCULES (Biogen Idec) and TvAb (Roche).

In some embodiments, Fc fusion molecules include to ScFv/Fc Fusions (Academic Institution), SCORPION (Emergent BioSolutions/Trubion, Zymogenetics/BMS) and Dual Affinity Retargeting Technology (Fc-DART) (MacroGenics).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based and domain antibodies include Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

### Polynucleotides, Vectors and Host Cells

The invention also provides for bispecific anti-TNF-α/IL-17A antibodies having certain HC1, LC1, HC2 and LC2 amino acid sequences, wherein the HC1, the LC1, the HC2 and the LC2 are encoded by certain polynucleotides. The polynucleotides may be a complementary deoxynucleic acid (cDNA), and may be codon optimized for expression in suitable host. Codon optimization is a well-known technology.

The invention also provides for an isolated polynucleotide encoding the HC1, the LC1, the HC2 and/or the LC2 of the bispecific anti-TNF-α/IL-17A antibodies of the invention. Certain exemplary polynucleotides are disclosed herein, however, other polynucleotides which, given the degeneracy of the genetic code or codon preferences in a given expression system, encode the antibodies of the invention are also within the scope of the invention.

The invention also provides for an isolated synthetic polynucleotide encoding the HC1 of SEQ ID NO: 5 or 7.

In some embodiments, the synthetic polynucleotide comprises the polynucleotide sequence of SEQ ID NO: 33 or 34.

The invention also provides for an isolated synthetic polynucleotide encoding the LC1 of SEQ ID NO: 6.

In some embodiments, the synthetic polynucleotide comprises the polynucleotide sequence of SEQ ID NO: 35.

The invention also provides for an isolated synthetic polynucleotide encoding the HC2 of SEQ ID NO: 8 or 10.

In some embodiments, the synthetic polynucleotide comprises the polynucleotide sequence of SEQ ID NO: 36 or 37.

The invention also provides for an isolated synthetic polynucleotide encoding the LC1 of SEQ ID NO: 9.

In some embodiments, the synthetic polynucleotide comprises the polynucleotide sequence of SEQ ID NO: 38.

The invention also provides a polynucleotide encoding the VH, or the VH and the VL of the anti-TNF-α antibody of the invention comprising the VH of SEQ ID NO: 11 and the VL of SEQ ID NO: 12.

In some embodiments, the polynucleotide comprises the polynucleotide sequence of SEQ ID NOs: 39 or 40.

DNA encoding HC1 of SEQ ID NO: 5 (TNF-α binding
HC of mAb 9762)
SEQ ID NO: 33
CAGGTACAGCTGGTGGAGTCTGGGGGAGGCGTGGTCCAGCCTGGGAGGTC
CCTGAGACTCTCCTGTGCAGCCTCTGGATTCATCTTCAGTAGCTATGCTA
TGCACTGGGTCCGCCAGGCACCAGGCAAAGGGCTGGAGTGGGTGGCATTT
ATGTCATATGATGGAAGCAATAAAAAGTACGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAGAGATCGA
GGAATAGCAGCAGGTGGAAACTACTACTACTACGGTATGGACGTCTGGGG
CCAAGGGACCACGGTCACCGTCTCCTCAGCCTCCACCAAGGGCCCATCGG
TCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGC
AGCTTGGGCACCCAGACCTACATCTGCAACGTGAATCACAAGCCCAGCAA
CACCAAGGTGGACAAGAAAGTTGAGCCCAAATCTTGTGACAAAACTCACA
CATGCCCACCGTGCCCAGCACCTGAACTCCTGGGGGGACCGTCAGTCTTC
CTCTTCCCCCCAAAACCCAAGGACACCCTCATGATCTCCCGGACCCCTGA
GGTCACATGCGTGGTGGTGGACGTGAGCCACGAAGACCCTGAGGTCAAGT
TCAACTGGTACGTGGACGGCGTGGAGGTGCATAATGCCAAGACAAAGCCG
CGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGG
CAGCCCCGAGAACCACAGGTGTACACCCTGCCCCCATCCCGGGAAGAGAT
GACCAAGAACCAGGTCAGCCTGACCTGCCTGGTCAAAGGCTTCTATCCCA
GCGACATCGCCGTGGAGTGGGAGAGCAATGGGCAGCCGGAGAACAACTAC
AAGACCACGCCTCCCGTGCTGGACTCCGACGGCTCCTTCTTCCTCTACAG
CCGGCTCACCGTGGACAAGAGCAGGTGGCAGCAGGGGAACGTCTTCTCAT
GCTCCGTGATGCATGAGGCTCTGCACAACCACTACACGCAGAAGAGCCTC
TCCCTGTCTCCGGGTAAA
DNA encoding HC1 of SEQ ID NO: 7 (TNF-α binding
HC of mAb 8759)
SEQ ID NO: 34
CAGGTACAGCTGGTGGAGTCTGGGGGAGGCGTGGTCCAGCCTGGGAGGTC
CCTGAGACTCTCCTGTGCAGCCTCTGGATTCATCTTCAGTAGCTATGCTA
TGCACTGGGTCCGCCAGGCACCAGGCAAAGGGCTGGAGTGGGTGGCATTT
ATGTCATATGATGGAAGCAATAAAAAGTACGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAGAGATCGA
GGAATAGCAGCAGGTGGAAACTACTACTACTACGGTATGGACGTCTGGGG
CCAAGGGACCACGGTCACCGTCTCCTCAGCCTCCACCAAGGGCCCATCGG
TCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGC
AGCTTGGGCACCCAGACCTACATCTGCAACGTGAATCACAAGCCCAGCAA
CACCAAGGTGGACAAGAAAGTTGAGCCCAAATCTTGTGACAAAACTCACA
CATGCCCACCGTGCCCAGCACCTGAACTCCTGGGGGGACCGTCAGTCTTC
CTCTTCCCCCCAAAACCCAAGGACACCCTCTACATCACCCGGGAACCTGA
GGTCACATGCGTGGTGGTGGACGTGAGCCACGAAGACCCTGAGGTCAAGT
TCAACTGGTACGTGGACGGCGTGGAGGTGCATAATGCCAAGACAAAGCCG
CGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGG
CAGCCCCGAGAACCACAGGTGTACACCCTGCCCCCATCCCGGGAAGAGAT
GACCAAGAACCAGGTCAGCCTGACCTGCCTGGTCAAAGGCTTCTATCCCA
GCGACATCGCCGTGGAGTGGGAGAGCAATGGGCAGCCGGAGAACAACTAC
AAGACCACGCCTCCCGTGCTGGACTCCGACGGCTCCTTCTTCCTCTACAG
CCGGCTCACCGTGGACAAGAGCAGGTGGCAGCAGGGGAACGTCTTCTCAT
GCTCCGTGATGCATGAGGCTCTGCACAACCACTACACGCAGAAGAGCCTC
TCCCTGTCTCCGGGTAAA
DNA encoding LC1 of SEQ ID NO: 6 (TNF-α binding
LC of mAb 9762 and mAb 8759)
SEQ ID NO: 35
GAAATTGTGTTGACACAGTCTCCAGCCACCCTGTCTTTGTCTCCAGGGGA
AAGAGCCACCCTCTCCTGCAGGGCCAGTCAGAGTGTTTACAGCTACTTAG
CCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGCGTAGCAACTGGCCTCCATTCACTTTCGGC
CCTGGGACCAAAGTGGATATCAAACGTACGGTGGCTGCACCATCTGTCTT
CATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCA
GGACAGCAAGGACAGCACCTACAGCCTCAGCAGCACCCTGACGCTGAGCA
AAGCAGACTACGAGAAACACAAAGTCTACGCCTGCGAAGTCACCCATCAG
GGCCTGAGCTCGCCCGTCACAAAGAGCTTCAACAGGGGAGAGTGT
DNA encoding HC2 of SEQ ID NO: 8 (IL-17A binding
HC of mAb 9762)
SEQ ID NO: 36
CAAGTGCAGCTGCTGGAGAGCGGCGGCGGCCTGGTGCAGCCCGGCGGCAG
CCTGCGGCTGAGCTGCGCCGCCAGCGGCTTCACCTTCAGCAGCTACGCCA
TGAGCTGGGTGCGGCAGGCCCCCGGCAAGGGCCTGGAGTGGGTGAGCACC
ATCAGCCTGACCAGCGGCTTCACCTACTACGCCGACAGCGTGAAGGGCCG
GTTCACCATCAGCCGGGACAACAGCAAGAACACCCTGTACCTGCAGATGA
ACAGCCTGCGGGCCGAGGACACCGCCGTGTACTACTGCGCCCGGCAGCTG
ACCCTGGACGTGTGGGGCCAGGGCACCCTGGTGACCGTGAGCAGCGCCTC
CACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTG
AATCACAAGCCCAGCAACACCAAGGTGGACAAGAAAGTTGAGCCCAAATC
TTGTGACAAAACTCACACATGCCCACCGTGCCCAGCACCTGAACTCCTGG
GGGGACCGTCAGTCTTCCTCTTCCCCCCAAAACCCAAGGACACCCTCATG
ATCTCCCGGACCCCTGAGGTCACATGCGTGGTGGTGGACGTGAGCCACGA
AGACCCTGAGGTCAAGTTCAACTGGTACGTGGACGGCGTGGAGGTGCATA
ATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCCC
CCATCCCGGGAAGAGATGACCAAGAACCAGGTCAGCCTGACCTGCCTGGT
CAAAGGCTTCTATCCCAGCGACATCGCCGTGGAGTGGGAGAGCAATGGGC
AGCCGGAGAACAACTACAAGACCACGCCTCCCGTGCTGGACTCCGACGGC
TCCTTCCTCCTCTACAGCAAGCTCACCGTGGACAAGAGCAGGTGGCAGCA
GGGGAACGTCTTCTCATGCTCCGTGATGCATGAGGCTCTGCACAACCACT
ACACGCAGAAGAGCCTCTCCCTGTCTCCGGGTAAA
DNA encoding HC2 of SEQ ID NO: 10 (IL-17A binding
HC of mAb 8759)
SEQ ID NO: 37
CAAGTGCAGCTGCTGGAGAGCGGCGGCGGCCTGGTGCAGCCCGGCGGCAG
CCTGCGGCTGAGCTGCGCCGCCAGCGGCTTCACCTTCAGCAGCTACGCCA
TGAGCTGGGTGCGGCAGGCCCCCGGCAAGGGCCTGGAGTGGGTGAGCACC
ATCAGCCTGACCAGCGGCTTCACCTACTACGCCGACAGCGTGAAGGGCCG
GTTCACCATCAGCCGGGACAACAGCAAGAACACCCTGTACCTGCAGATGA
ACAGCCTGCGGGCCGAGGACACCGCCGTGTACTACTGCGCCCGGCAGCTG
ACCCTGGACGTGTGGGGCCAGGGCACCCTGGTGACCGTGAGCAGCGCCTC
CACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTG
AATCACAAGCCCAGCAACACCAAGGTGGACAAGAAAGTTGAGCCCAAATC
TTGTGACAAAACTCACACATGCCCACCGTGCCCAGCACCTGAACTCCTGG
GGGGACCGTCAGTCTTCCTCTTCCCCCCAAAACCCAAGGACACCCTCTAC
ATCACCCGGGAACCTGAGGTCACATGCGTGGTGGTGGACGTGAGCCACGA
AGACCCTGAGGTCAAGTTCAACTGGTACGTGGACGGCGTGGAGGTGCATA
ATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCCC
CCATCCCGGGAAGAGATGACCAAGAACCAGGTCAGCCTGACCTGCCTGGT
CAAAGGCTTCTATCCCAGCGACATCGCCGTGGAGTGGGAGAGCAATGGGC
AGCCGGAGAACAACTACAAGACCACGCCTCCCGTGCTGGACTCCGACGGC
TCCTTCCTCCTCTACAGCAAGCTCACCGTGGACAAGAGCAGGTGGCAGCA
GGGGAACGTCTTCTCATGCTCCGTGATGCATGAGGCTCTGCACAACCACT
ACACGCAGAAGAGCCTCTCCCTGTCTCCGGGTAAA
DNA encoding LC2 of SEQ ID NO: 9 (IL-17A binding
LC of mAb 9762 and mAb 8759)
SEQ ID NO: 38
CAGAGCGTGCTGACCCAGCCCCCCAGCGTGAGCGTGGCCCCCGGCCAGAC
CGCCCGGATCAGCTGCAGCGGCGACAACCTGGGCGACAAGTACGCCAACT
GGTACCAGCAGAAGCCCGGCCAGGCCCCCGTGCTGGTGATCTACGACGAC
ATCGACCGGCCCAGCGGCATCCCCGAGCGGTTCAGCGGCAGCAACAGCGG
CAACACCGCCACCCTGACCATCAGCGGCACCCAGGCCGAGGACGAGGCCG
ACTACTACTGCGGCAGCTACGACTTCTTCCTGGGCATGATCGTGTTCGGC
GGCGGCACCAAGCTGACCGTGCTGGGTCAGCCCAAGGCTGCACCCAGTGT
CACTCTGTTCCCGCCCTCCTCTGAGGAGCTTCAAGCCAACAAGGCCACAC
TGGTGTGTCTCATAAGTGACTTCTACCCGGGAGCCGTGACAGTGGCCTGG
AAGGCCGATAGCAGCCCCGTCAAGGCGGGAGTGGAGACCACCACACCCTC
CAAACAAAGCAACAACAAGTACGCGGCCAGCAGCTATCTGAGCCTGACGC
CTGAGCAGTGGAAGTCCCACAGAAGCTACAGCTGCCAGGTCACGCATGAA
GGGAGCACCGTGGAGAAGACAGTGGCCCCTACAGAATGTTCA
DNA encoding VH of SEQ ID NO: 11 (TNF-α binding
VH of mAb 9762)
SEQ ID NO: 39
CAGGTACAGCTGGTGGAGTCTGGGGGAGGCGTGGTCCAGCCTGGGAGGTC
CCTGAGACTCTCCTGTGCAGCCTCTGGATTCATCTTCAGTAGCTATGCTA
TGCACTGGGTCCGCCAGGCACCAGGCAAAGGGCTGGAGTGGGTGGCATTT
ATGTCATATGATGGAAGCAATAAAAAGTACGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAGAGATCGA
GGAATAGCAGCAGGTGGAAACTACTACTACTACGGTATGGACGTCTGGGG
CCAAGGGACCACGGTCACCGTCTCCTCA
DNA encoding VL of SEQ ID NO: 12 (TNF-α binding
VL of mAb 9762)
SEQ ID NO: 40
GAAATTGTGTTGACACAGTCTCCAGCCACCCTGTCTTTGTCTCCAGGGGA
AAGAGCCACCCTCTCCTGCAGGGCCAGTCAGAGTGTTTACAGCTACTTAG
CCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGCGTAGCAACTGGCCTCCATTCACTTTCGGC
CCTGGGACCAAAGTGGATATCAAA

The invention also provides for a vector comprising the polynucleotide of the invention.

Such vectors may be plasmid vectors, viral vectors, vectors for baculovirus expression, transposon based vectors or any other vector suitable for introduction of the synthetic polynucleotide of the invention into a given organism or genetic background by any means. The DNA segments encoding immunoglobulin chains may be operably linked to control sequences in the expression vector(s) that ensure the expression of immunoglobulin polypeptides. Such control sequences include signal sequences, promoters (e.g. naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed with the desired DNA sequences.

Suitable promoter and enhancer elements are known in the art. For expression in a eukaryotic cell, exemplary promoters include light and/or heavy chain immunoglobulin gene promoter and enhancer elements, cytomegalovirus immediate early promoter, herpes simplex virus thymidine kinase promoter, early and late SV40 promoters, promoter present in long terminal repeats from a retrovirus, mouse metallothionein-I promoter, tetracycline-inducible promoter, and various art-known tissue specific promoters. Selection of the appropriate vector and promoter is well known.

Large numbers of suitable vectors and promoters are known. Many are commercially available for generating recombinant constructs. Exemplary vectors are bacterial vectors pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden), and eukaryotic vectors pWLneo, pSV2cat, pOG44, PXR1, pSG (Stratagene) pSVK3, pBPV, pMSG, pSVL (Pharmacia), pEE6.4 (Lonza) and pEE12.4 (Lonza).

Another embodiment of the invention is a host cell comprising one or more vectors of the invention. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells.

Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHOK1SV (Lonza Biologics, Walkersville, Md.), Potelligent® CHOK2SV (Lonza), CHO-Kl (ATCC CRL-61) or DG44.

The invention also provides for a method of producing the antibody of the invention comprising culturing the host cell of the invention in conditions that the antibody is expressed, and recovering the antibody produced by the host cell. Methods of making antibodies and purifying them are well known. Once synthesized (either chemically or recombinantly), the whole antibodies, their dimers, individual light and/or heavy chains, or other antibody fragments such as VH and/or VL, may be purified according to standard procedures, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). The antibody of the invention may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the antibody of the invention.

The polynucleotides encoding certain HC, LC, VH and/or VL, sequences of the invention described herein may be incorporated into vectors using standard molecular biology methods. Host cell transformation, culture, antibody expression and purification are done using well known methods.

The invention also provides for a method of producing the isolated bispecific anti-TNF-α/IL-17A antibody of the invention, comprising:
combining an isolated monospecific bivalent anti-TNF-α antibody comprising two heavy chains of SEQ ID NO: 5 or two heavy chains of SEQ ID NO: 7 and two light chains of SEQ ID NO: 6 and an isolated monospecific bivalent anti-IL-17A antibody comprising two heavy chains of SEQ ID NO: 8 or two heavy chains of SEQ ID NO: 10 and two light chains of SEQ ID NO: 9 in a mixture of about 1:1 molar ratio;
introducing a reducing agent into the mixture;
incubating the mixture about ninety minutes to about six hours;
removing the reducing agent; and
purifying
the bispecific anti-TNF-α/IL-17A antibody that comprises a first heavy chain of SEQ ID NO: 5 and a second heavy chain of SEQ ID NO: 8, a first light chain of SEQ ID NO: 6 and a second light chain of SEQ ID NO: 9, wherein the first heavy chain of SEQ ID NO: 5 pairs with the first light chain of SEQ ID NO: 6 to form a first binding domain that specifically binds TNF-α, and the second heavy chain of SEQ ID NO: 8 pairs with the second light chain of SEQ ID NO: 9 to form a second binding domain that specifically binds IL-17A; or
   purifying the bispecific anti-TNF-α/IL-17A antibody that comprises a first heavy chain of SEQ ID NO: 7 and a second heavy chain of SEQ ID NO: 10, a first light chain of SEQ ID NO: 6 and a second light chain of SEQ ID NO: 9, wherein the first heavy chain of SEQ ID NO: 7 pairs with the first light chain of SEQ ID NO: 6 to form the first binding domain that specifically binds TNF-α, and the second heavy chain of SEQ ID NO: 10 pairs with the second light chain of SEQ ID NO: 9 to form the second binding domain that specifically binds IL-17A.

### Pharmaceutical Compositions/Administration

The invention also provides for pharmaceutical compositions comprising the bispecific anti-TNF-α/IL-17A antibodies of the invention or the anti-TNF-α antibodies of the invention and a pharmaceutically acceptable carrier. For therapeutic use, the antibodies of the invention may be prepared as pharmaceutical compositions containing an effective amount of the antibody as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies of the invention in such pharmaceutical formulations may vary, from less than about 0.5%, usually to at least about 1% to as much as 15 or 20% by weight and may be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21′ Edition, Troy, D.B. ed., Lipincott Williams and Wilkins, Philadelphia, PA 2006, Part 5, Pharmaceutical Manufacturing pp 691-1092, See especially pp. 958-989.

An exemplary pharmaceutical composition comprises 40 mg/mL antibody, 10 mM histidine, 8.5% (w/v) sucrose and 0.04% (w/v) Polysorbate 80 at pH 5.8.

The invention also provides for a pharmaceutical composition comprising 40 mg/ml of the bispecific anti-TNF-α/IL-17A antibody comprising the HC1 and the LC1 of SEQ ID NOs: 5 and 6, respectively, and the HC2 and the LC2 of SEQ ID NOs: 8 and 9, respectively, 10 mM histidine, 8.5% (w/v) sucrose and 0.04% (w/v) Polysorbate 80 at pH 5.8.

The invention also provides for a pharmaceutical composition comprising 40 mg/ml of a bispecific anti-TNF-α/IL-17A antibody comprising the HC1 and the LC1 of SEQ ID NOs: 7 and 6, respectively, and the HC2 and the LC2 of SEQ ID NOs: 10 and 9, respectively, 10 mM histidine, 8.5% (w/v) sucrose and 0.04% (w/v) Polysorbate 80 at pH 5.8.

The invention also provides for a pharmaceutical composition comprising the anti-TNF-α antibody comprising the VH of SEQ ID NO: 5 and the VL of SEQ ID NO: 6.

The mode of administration for therapeutic use of the antibodies of the invention may be any suitable route that delivers the antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

The antibodies of the invention may be administered to a subject by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours.

The dose given to a subject is sufficient to alleviate or at least partially arrest the disease being treated (“therapeutically effective amount”) and may be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 0.1, 0.2, 0.3, 0.4, 0.5, 0.6, 0.7, 0.8, 0.9, 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

The dose of the antibodies of the invention given to a subject may be about 0.1 mg/kg to 10 mg/kg via intravenous administration.

The dose of the antibodies of the invention given to a subject may be about 0.1 mg/kg to 10 mg/kg via subcutaneous administration.

The dose of the antibodies of the invention given to a subject may be about 0.1 mg/kg via intravenous administration.

The dose of the antibodies of the invention given to a subject may be about 0.1 mg/kg via subcutaneous administration.

The dose of the antibodies of the invention given to a subject may be about 0.3 mg/kg via intravenous administration.

The dose of the antibodies of the invention given to a subject may be about 0.3 mg/kg via subcutaneous administration.

The dose of the antibodies of the invention given to a subject may be about 1.0 mg/kg via intravenous administration.

The dose of the antibodies of the invention given to a subject may be about 1.0 mg/kg via subcutaneous administration.

The dose of the antibodies of the invention given to a subject may be about 3.0 mg/kg via intravenous administration.

The dose of the antibodies of the invention given to a subject may be about 3.0 mg/kg via subcutaneous administration.

The dose of the antibodies of the invention given to a subject may be about 10.0 mg/kg via intravenous administration.

The dose of the antibodies of the invention given to a subject may be about 10.0 mg/kg via subcutaneous administration.

A fixed unit dose of the antibodies of the invention may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat the patient, but 9, 10, 11, 12, 13, 14, 15, 16, 17, 18, 19, 20 or more doses may be given.

The administration of the antibodies of the invention described herein may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the antibodies of the invention described herein may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion. Alternatively, the antibodies of the invention described herein may be administered at between 0.1 mg/kg to about 10 mg/kg at weekly interval for 17 weeks.

For example, the antibodies of the invention may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The antibodies of the invention described herein may also be administered prophylactically in order to reduce the risk of developing an inflammatory disease such as RA, psoriatic arthritis or psoriasis, delay the onset of the occurrence of an event in progression of the inflammatory disease such as RA, psoriatic arthritis or psoriasis.

The antibodies of the invention may be lyophilized for storage and reconstituted in a suitable carrier prior to use. This technique has been shown to be effective with conventional protein preparations and well known lyophilization and reconstitution techniques can be employed.

The antibodies of the invention may be supplied as a sterile, frozen liquid in a glass vial with stopper and aluminum seal with flip-off cap. Each vial may contain 3.3 mL of a 50 mg/mL solution of the antibody (including a 10% overfill) in a formulation of 10 mM histidine, 8.5% (w/v) sucrose, and 0.04% (w/v) Polysorbate 80 at pH 5.8. Vials may contain no preservatives and thus may be for single use. Vials may be stored frozen and protected from light. To prepare the antibody for IV administration, the antibody formulation may be filtered with a 0.22 micron filter before being diluted in sterile diluent. Diluted antibody at volumes up to approximately 100 mL may be administered by IV infusion over a period of at least 30 minutes using an in-line 0.22 micron filter.

Alternatively, the antibody may be administered as 1 or 2 subcutaneous injections of 50 mg/mL antibody in about 3.3 mL. The subcutaneous injection site may be within the abdominal area.

### Methods and Uses

The bispecific anti-TNF-α/IL-17A and the anti-TNF-α antibodies of the invention have in vitro and in vivo diagnostic, as well as therapeutic and prophylactic utilities. For example, the antibodies of the invention described herein may be administered to cells in culture, in vitro or ex vivo, or to a subject to treat, prevent, and/or diagnose a variety of disorders, such as an inflammatory disease.

The bispecific anti-TNF-α/IL-17A antibodies of the invention may be useful for treating or preventing rheumatoid arthritis or other inflammatory disorders such as psoriasis, psoriatic arthritis, lupus (systemic lupus erythematosus, SLE, or lupus nephritis), ankylosing spondylitis, Crohn's disease, ulcerative colitis and juvenile idiopathic arthritis, general inflammatory diseases (e.g. conjunctivitis).

The bispecific anti-TNF-α/IL-17A antibodies of the invention may be useful in treating or preventing rheumatoid arthritis in patients exhibiting elevated TNF-α and/or IL-17A.

The bispecific anti-TNF-α/IL-17A antibodies of the invention may be useful in treating or preventing rheumatoid arthritis in patients who are non-responsive to anti-TNF-α treatment.

The invention provides for a use of the bispecific anti-TNF-α/IL-17A antibodies of the invention for treating or preventing rheumatoid arthritis.

The invention also provides for a use of the bispecific anti-TNF-α/IL-17A antibodies of the invention for the treatment or prevention of rheumatoid arthritis in patients exhibiting elevated TNF-α and/or IL-17 or in patients who have been determined to have elevated TNF-α and/or IL-17.

The invention also provides for a use of the bispecific anti-TNF-α/IL-17A antibodies of the invention for treating or preventing rheumatoid arthritis in patients who are non-responsive to anti-TNF-α treatment.

The invention further provides for a use of the bispecific anti-TNF-α/IL-17A antibodies of the invention for preventing or treating rheumatoid arthritis.

The invention further provides for a use of the bispecific anti-TNF-α/IL-17A antibodies of the invention for preventing or treating an inflammatory disorder such as psoriatic arthritis, psoriasis, lupus (systemic lupus erythematosus, SLE, or lupus nephritis), ankylosing spondylitis, Crohn's disease, ulcerative colitis and juvenile idiopathic arthritis, and general inflammatory diseases such as conjunctivitis.

The invention provides bispecific anti-TNF-α/IL-17A antibodies as described herein for use in a method of treatment.

The invention provides for a method of treating TNF-α-mediated inflammatory disease, comprising administering to a subject in need thereof a therapeutically effective amount of the bispecific anti-TNF-α/IL-17A antibody of the invention for a time sufficient to treat TNF-α-mediated inflammatory disease.

The invention provides for a method of treating TNF-α-mediated inflammatory disease, comprising administering to a subject in need thereof a therapeutically effective amount of the anti-TNF-α antibody of the invention for a time sufficient to treat TNF-α-mediated inflammatory disease.

“TNF-α-mediated inflammatory disease” refers to a disease where TNF-α has been shown to play a pathophysiological role. Exemplary TNF-αa-mediated inflammatory diseases are autoimmune diseases, inflammatory bowel disease, Crohn's disease, ulcerative colitis, arthritis, rheumatoid arthritis, psoriatic arthritis, ankylosing spondylitis, spondyloarthritis, psoriasis, juvenile psoriasis, juvenile idiopathic arthritis, axial Bechet's disease, Hidradentis suppurativa, uveitis, asthma, sepsis, lupus erythematosus, cutaneous infection, cachexia, Wegener's granulomatosis, pulmonary fibrosis, chronic obstructive pulmonary disease, heart failure, Kawasaki disease, fascular sarcoidosis, type 1 diabetes, ischemia, infarction, anal fistula, ichthyosis and seborrhea.

The invention provides for a method of treating IL-17A-mediated inflammatory disease, comprising administering to a subject in need thereof a therapeutically effective amount of the bispecific anti-TNF-α/IL-17A antibody of the invention for a time sufficient to treat IL-17A-mediated inflammatory disease.

“IL-17A-mediated inflammatory disease” refers to a disease where IL-17A has been shown to play a pathophysiological role. Exemplary IL-17A-mediated diseases are autoimmune diseases, inflammatory bowel disease, Crohn's disease, ulcerative colitis, arthritis, rheumatoid arthritis, psoriatic arthritis, juvenile idiopathic arthritis, ankylosing spondylitis, spondyloarthritis, psoriasis, juvenile psoriasis, axial Bechet's disease, Hidradentis suppurativa, uveitis, asthma, sepsis, lupus, lupus erythematosus, cutaneous infection, cachexia, Wegener's granulomatosis, pulmonary fibrosis, chronic obstructive pulmonary disease, heart failure, Kawasaki disease, fascular sarcoidosis, type 1 diabetes, ischemia, infarction, anal fistula, ichthyosis, seborrhea and acne.

The invention provides for a method of treating TNF-α-mediated autoimmune disease, comprising administering to a subject in need thereof a therapeutically effective amount of the bispecific anti-TNF-α/IL-17A antibody of the invention for a time sufficient to treat TNF-α-mediated autoimmune disease.

The invention provides for a method of treating IL-17A-mediated autoimmune disease, comprising administering to a subject in need thereof a therapeutically effective amount of the bispecific anti-TNF-α/IL-17A antibody of the invention for a time sufficient to treat IL-17A-mediated autoimmune disease.

The TNF-α-mediated inflammatory disease may be rheumatoid arthritis, systemic juvenile idiopathic arthritis, Grave's disease, Hashimoto's thyroiditis, myasthenia gravis, multiple sclerosis, systemic lupus erythematosus, Type 1 Diabetes, psoriasis or psoriatic arthritis.

The invention also provides for a method of treating rheumatoid arthritis (RA), comprising administering to a subject in need thereof a therapeutically effective amount of the bispecific anti-TNF-α/IL-17A antibody of the invention for a time sufficient to treat RA.

The invention also provides for a method of treating psoriasis, comprising administering to a subject in need thereof a therapeutically effective amount of the bispecific anti-TNF-α/IL-17A antibody of the invention for a time sufficient to treat psoriasis.

The invention also provides for a method of treating psoriatic arthritis, comprising administering to a subject in need thereof a therapeutically effective amount of the bispecific anti-TNF-α/IL-17A antibody of the invention for a time sufficient to treat psoriatic arthritis.

The invention also provides for a method of treating rheumatoid arthritis (RA), comprising administering to a subject in need thereof a therapeutically effective amount of the bispecific anti-TNF-α/IL-17A antibody comprising the HC1, the LC1, the HC2 and the LC2 of SEQ ID NOs: 5, 6, 8 and 9, respectively, for a time sufficient to treat RA.

The invention also provides for a method of treating rheumatoid arthritis (RA), comprising administering to a subject in need thereof a therapeutically effective amount of the bispecific anti-TNF-α/IL-17A antibody comprising the HC1, the LC1, the HC2 and the LC2 of SEQ ID NOs: 7, 6, 10 and 9, respectively, for a time sufficient to treat RA.

The invention also provides for a method of treating psoriasis, comprising administering to a subject in need thereof a therapeutically effective amount of the bispecific anti-TNF-α/IL-17A antibody comprising the HC1, the LC1, the HC2 and the LC2 of SEQ ID NOs: 5, 6, 8 and 9, respectively, for a time sufficient to treat psoriasis.

The invention provides a method of treating psoriasis, comprising administering to a subject in need thereof a therapeutically effective amount of the bispecific anti-TNF-α/IL-17A antibody comprising the HC1, the LC1, the HC2 and the LC2 of SEQ ID NOs: 7, 6, 10 and 9, respectively, for a time sufficient to treat psoriasis.

The invention also provides for a method of treating psoriatic arthritis, comprising administering to a subject in need thereof a therapeutically effective amount of the bispecific anti-TNF-α/IL-17A antibody comprising the HC1, the LC1, the HC2 and the LC2 of SEQ ID NOs: 5, 6, 8 and 9, respectively, for a time sufficient to treat psoriatic arthritis.

The invention also provides for a method of treating psoriatic arthritis, comprising administering to a subject in need thereof a therapeutically effective amount of the bispecific anti-TNF-α/IL-17A antibody comprising the HC1, the LC1, the HC2 and the LC2 of SEQ ID NOs: 7, 6, 10 and 9, respectively, for a time sufficient to treat psoriatic arthritis.

The “therapeutically effective amount” of the bispecific anti-TNF-α/IL-17A antibodies or the anti-TNF-α antibodies of the invention effective in the treatment of a disease may be determined by standard research techniques. For example, in vitro assays may be employed to help identify optimal dosage ranges. Optionally, the dosage of the bispecific anti-TNF-α/IL-17A antibody of the invention that may be effective in the treatment of a disease such as arthritis or rheumatoid arthritis may be determined by administering the bispecific anti-TNF-α/IL-17A antibody to relevant animal models well known in the art. Selection of a particular effective dose may be determined (e.g., via clinical trials) by those skilled in the art based upon the consideration of several factors. Such factors include the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems. The antibodies of the invention may be tested for their efficacy and effective dosage using any of the models described herein.

### Combination Therapies

The bispecific anti-TNF-α/IL-17A antibodies of the invention described herein may be administered in combination with a second therapeutic agent.

“In combination with” refers to administering of the antibodies of the invention described herein and a second therapeutic agent concurrently as single agents or sequentially as single agents in any order. In general, each agent will be administered at a dose and/or on a time schedule determined for that agent.

The bispecific anti-TNF-α/IL-17A antibodies of the invention may be administered in combination with any known therapies for autoimmune diseases, including any agent or combination of agents that are known to be useful, or which have been used or are currently in use, for treatment of autoimmune diseases. Such therapies and therapeutic agents include surgery or surgical procedures (e.g. splenectomy, lymphadenectomy, thyroidectomy, plasmapheresis, leukophoresis, cell, tissue, or organ transplantation, intestinal procedures, organ perfusion, and the like), radiation therapy, therapy such as steroid therapy and non-steroidal therapy, hormone therapy, cytokine therapy, therapy with dermatological agents (for example, topical agents used to treat skin conditions such as allergies, contact dermatitis, and psoriasis), immunosuppressive therapy, and other anti-inflammatory monoclonal antibody therapy.

In some embodiments of the invention, the bispecific anti-TNF-α/IL-17A antibodies of the invention are administered in combination with a second therapeutic agent. Exemplary second therapeutic agents are corticosteroids, nonsteroidal anti-inflammatory drugs (NSAIDs), salicylates, hydroxychloroquine, sulfasalazine, cytotoxic drugs, immunosuppressive drugs immunomodulatory antibodies, methotrexate, cyclophosphamide, mizoribine, chlorambucil, cyclosporine, tacrolimus (FK506; ProGrafrM), mycophenolate mofetil, and azathioprine (6-mercaptopurine), sirolimus (rapamycin), deoxyspergualin, leflunomide and its malononitriloamide analogs; anti-CTLA4 antibodies and Ig fusions, anti-B lymphocyte stimulator antibodies (e.g., LYMPHOSTAT-BTM) and CTLA4-Ig fusions (BLyS-1 g), anti-CD80 antibodies, anti-T cell antibodies such as anti-CD3 (OKT3), anti-CD4, corticosteroids such as, for example, clobetasol, halobetasol, hydrocortisone, triamcinolone, betamethasone, fluocinole, fluocinonide, prednisone, prednisolone, methylprednisolone; non-steroidal anti-inflammatory drugs (NSAIDs) such as, for example, sulfasalazine, medications containing mesalamine (known as 5-ASA agents), celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulindac, and tolmetin; phosphodiesterase-4 inhibitors, anti-TNFα antibodies infliximab (REMICADE®), golimumab (SIMPONI®) and adalimumab (HUMIRA®), thalidomide or its analogs such as lenalidomide.

Treatment effectiveness or RA may be assessed using effectiveness as measured by clinical responses defined by the American College of Rheumatology criteria, the European League of Rheumatism criteria, or any other criteria. See for example, Felson et al., (1995) Arthritis Rheum 38:727-35 and van Gestel et al., (1996) Arthritis Rheum 39:34-40.

The bispecific anti-TNF-α/IL-17A antibodies in the methods of the invention described herein, may be lyophilized for storage and reconstituted in a suitable carrier prior to use. This technique has been shown to be effective with conventional protein preparations and well known lyophilization and reconstitution techniques can be employed.

The bispecific anti-TNF-α/IL-17A antibodies in the methods of the invention described herein may be administered in combination with a second therapeutic agent simultaneously, sequentially or separately.

The second therapeutic agent may be a corticosteroid, an antimalarial drug, an immunosuppressant, a cytotoxic drug, or a B-cell modulator.

In some embodiments, the second therapeutic agent is prednisone, prednisolone, methylprednisolone, deflazcort, hydroxychloroquine, azathioprine, methotrexate, cyclophosphamide, mycophenolate mofetil (MMF), mycophenolate sodium, cyclosporine, leflunomide, tacrolimus, RITUXAN® (rituximab), or BENLYSTA® (belimumab).

In some embodiments, the second therapeutic agent is corticosteroids, nonsteroidal anti-inflammatory drugs (NSAIDs), salicylates, hydroxychloroquine, sulfasalazine, cytotoxic drugs, immunosuppressive drugs immunomodulatory antibodies, methotrexate, cyclophosphamide, mizoribine, chlorambucil, cyclosporine, tacrolimus (FK506; ProGrafrM), mycophenolate mofetil, and azathioprine (6-mercaptopurine), sirolimus (rapamycin), deoxyspergualin, leflunomide and its malononitriloamide analogs; anti-CTLA4 antibodies and Ig fusions, anti-B lymphocyte stimulator antibodies (e.g., LYMPHOSTAT-BTM) and CTLA4-Ig fusions (BLyS-1 g), anti-CD80 antibodies, anti-T cell antibodies such as anti-CD3 (OKT3), anti-CD4, corticosteroids such as, for example, clobetasol, halobetasol, hydrocortisone, triamcinolone, betamethasone, fluocinole, fluocinonide, prednisone, prednisolone, methylprednisolone; non-steroidal anti-inflammatory drugs (NSAIDs) such as, for example, sulfasalazine, medications containing mesalamine (known as 5-ASA agents), celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulindac, and tolmetin; phosphodiesterase-4 inhibitors, anti-TNFα antibodies REMICADE® (infliximab), SIMPONI® (golimumab) and HUMIRA® (adalimumab), thalidomide or its analogs such as lenalidomide.

### Diagnostic Uses and Kits

### Kits

The invention also provides for a kit comprising the bispecific anti-TNF-α/IL-17A antibody of the invention.

The kit may be used for therapeutic uses and as diagnostic kits.

The kit may be used to detect the presence of TNF-α, IL-17A or TNF-α and IL-17A in a sample.

In some embodiments, the kit comprises the bispecific anti-TNF-α/IL-17A antibodies of the invention and reagents for detecting the antibody. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antibody of the invention in a container and instructions for use of the kit.

In some embodiments, the antibody in the kit is labeled.

The invention also provides for a kit comprising the bispecific anti-TNF-α/IL-17A antibody comprising the HC1, the LC1, the HC2 and the LC2 of SEQ ID NOs: 5, 6, 8 and 9, respectively.

The invention also provides for a kit comprising the bispecific anti-TNF-α/IL-17A antibody comprising the HC1, the LC1, the HC2 and the LC2 of SEQ ID NOs: 7, 6, 10 and 9, respectively.

Methods of detecting TNF-α, IL-17A or TNF-α and IL-17A

The invention also provides for a method of detecting TNF-α, IL-17A or TNF-αand IL-17A in a sample, comprising obtaining the sample, contacting the sample with the bispecific anti-TNF-α/IL-17A antibody of the invention, and detecting the antibody bound to detecting TNF-α, IL-17A or TNF-α and IL-17A in the sample.

In some embodiments described herein, the sample may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like.

The antibodies of the invention described herein bound to TNF-α, IL-17A or TNF-α and IL-17A may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antibodies of the invention may be used in a variety of assays to detect TNF-α, IL-17A or TNF-α and IL-17A in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

The invention will now be described with specific, non-limiting examples.

### EXAMPLE 1

### Generation of Bispecific Anti-TNF-α/IL-17A Antibodies

Select monospecific anti-TNF-α and anti-IL-17A antibodies were expressed as IgG1/κ (G1m(17) allotype). Substitutions were made at positions 405 and 409 (EU numbering) in the monospecific antibodies to promote subsequent in vitro arm exchange and formation of the bispecific antibodies. The IgG1 anti-TNF-α antibodies were engineered to have a K409R substitution, and the anti-IL-17A antibodies were engineered to have a F405L substitution to promote arm exchange and generation the bispecific antibodies. In addition to position 405 and 409 substitutions, the IgG1 mAbs were optionally engineered to have M252Y/S254T/T256E (EU numbering) substitutions to increase half-life of the resulting mAb (referred to as “YTE” in the specification).

The monospecific antibodies were expressed and purified using standard methods using a Protein A column (HiTrap MabSelect SuRe column). After elution, the pools were dialyzed into D-PBS, pH 7.2.

Bispecific anti-TNF-α/IL-17A antibodies were generated by combining a monospecific anti-TNF-α mAb and a monospecific anti-IL-17A mAb in in vitro Fab arm exchange as described in Int. Patent Publ. No. WO2011/131746. Briefly, at about 1-20 mg/ml at a molar ratio of 1:1 of each antibody in PBS, pH 7-7.4 and 75 mM 2-mercaptoethanolamine (2-MEA) was mixed together and incubated at 25-37° C. for 2-6 h, followed by removal of the 2-MEA via dialysis, diafiltration, tangential flow filtration and/or spinned cell filtration using standard methods.

The bispecific antibodies were further purified after the in vitro Fab-arm exchange using hydrophobic interaction chromatography to minimize residual parental anti-TNF-α and anti-IL-17A antibodies using standard methods.

The SEQ ID NOs: for the HC and the LC amino acid sequences of the parental monospecific (mAb 9809, mAb 6696, mAb 4782 and mAb 7206) and the generated bispecific anti-TNF-α/IL-17A antibodies (mAb 9762 and mAb 8759) are shown in Table 3. The VH and the VL SEQ ID NOs: for the amino acid sequences of the bispecific antibodies mAb 9762 and mAb 8759 are shown in Table 4. Table 5 shows the amino acid sequences. The VH and the VL sequences forming the TNF-α binding domain in the antibodies was derived from golimumab (SIMPONI®), except for a substitution N43K in the VH. The VH and the VL sequences forming the IL-17A binding domain in the antibodies were those of mAb 6785 described in U.S. Pat. No. 8,519,107. The N43K substitution in the antibody had no effect on the activity of the antibody when compared to golimumab.

TABLE 3

| | SEQ ID NO: | | | |
|---|---|---|---|---|
| | TNF-α binding arm | | IL-17A binding arm | |
| mAb (isotype) | HC | LC | HC | LC |
| 9809 (IgG1, K409R) | 5 | 6 | | |
| 6696 (IgG1, YTE*) | 7 | 6 | | |
| 4782 (IgG1, F405L) | | | 8 | 9 |
| 7206 (IgG1, YTE*) | | | 10 | 9 |
| 9762 (IgG1) | 5 | 6 | 8 | 9 |
| 8759 (IgG1, YTE*) | 7 | 6 | 10 | 9 |

*YTE: M252Y/S254T/T256E substitution;
HC: heavy chain;
LC: light chain

TABLE 4

| | SEQ ID NO: | | | |
|---|---|---|---|---|
| | TNF-α binding arm | | IL-17A binding arm | |
| mAb | VH | VL | VH | VL |
| 9762 (IgG1) | 11 | 12 | 13 | 14 |
| 8759 (IgG1, YTE*) | 11 | 12 | 13 | 14 |

*YTE: M252Y/5254T/T256E substitution

TABLE 5

| SEQ | |
|---|---|
| ID NO: | Amino acid sequence |
| 5 | QVQLVESGGGVVQPGRSLRLSCAASGFIFSSYAMHWVRQAPGKGLEW |
| | VAFMSYDGSNKKYADSVKGRFTISRDNSKNTLYLQMNSLRAEDTAVY |
| | YCARDRGIAAGGNYYYYGMDVWGQGTTVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPPC |
| | PAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFY |
| | PSDIAVEWESNGQPENNYKTTPPVLDSDGSFFLYSRLTVDKSRWQQGN |
| | VFSCSVMHEALHNHYTQKSLSLSPGK |
| 6 | EIVLTQSPATLSLSPGERATLSCRASQSVYSYLAWYQQKPGQAPRLLIY |
| | DASNRATGIPARFSGSGSGTDFTLTISSLEPEDFAVYYCQQRSNWPPFTF |
| | GPGTKVDIKRTVAAPSVFIFPPSDEQLKSGTASVVCLLNNFYPREAKVQ |
| | WKVDNALQSGNSQESVTEQDSKDSTYSLSSTLTLSKADYEKHKVYAC |
| | EVTHQGLSSPVTKSFNRGEC |
| 7 | QVQLVESGGGVVQPGRSLRLSCAASGFIFSSYAMHWVRQAPGKGLEW |
| | VAFMSYDGSNKKYADSVKGRFTISRDNSKNTLYLQMNSLRAEDTAVY |
| | YCARDRGIAAGGNYYYYGMDVWGQGTTVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPPC |
| | PAPELLGGPSVFLFPPKPKDTLYITREPEVTCVVVDVSHEDPEVKFNWY |
| | VDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFY |
| | PSDIAVEWESNGQPENNYKTTPPVLDSDGSFFLYSRLTVDKSRWQQGN |
| | VFSCSVMHEALHNHYTQKSLSLSPGK |
| 8 | QVQLLESGGGLVQPGGSLRLSCAASGFTFSSYAMSWVRQAPGKGLEW |
| | VSTISLTSGFTYYADSVKGRFTISRDNSKNTLYLQMNSLRAEDTAVYY |
| | CARQLTLDVWGQGTLVTVSSASTKGPSVFPLAPSSKSTSGGTAALGCL |
| | VKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISK |
| | AKGQPREPQVYTLPPSREEMTKNQVSLTCLVKGFYPSDIAVEWESNGQ |
| | PENNYKTTPPVLDSDGSFLLYSKLTVDKSRWQQGNVFSCSVMHEALH |
| | NHYTQKSLSLSPGK |
| 9 | QSVLTQPPSVSVAPGQTARISCSGDNLGDKYANWYQQKPGQAPVLVI |
| | YDDIDRPSGIPERFSGSNSGNTATLTISGTQAEDEADYYCGSYDFFLGMI |
| | VFGGGTKLTVLGQPKAAPSVTLFPPSSEELQANKATLVCLISDFYPGAV |
| | TVAWKADSSPVKAGVETTTPSKQSNNKYAASSYLSLTPEQWKSHRSY |
| | SCQVTHEGSTVEKTVAPTECS |
| 10 | QVQLLESGGGLVQPGGSLRLSCAASGFTFSSYAMSWVRQAPGKGLEW |
| | VSTISLTSGFTYYADSVKGRFTISRDNSKNTLYLQMNSLRAEDTAVYY |
| | CARQLTLDVWGQGTLVTVSSASTKGPSVFPLAPSSKSTSGGTAALGCL |
| | VKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLF |
| | PPKPKDTLYITREPEVTCVVVDVSHEDPEVKFNWYVDGVEVHNAKTK |
| | PREEQYNSTYRVVSVLTVLHQDWLNGKEYKCKVSNKALPAPIEKTISK |
| | AKGQPREPQVYTLPPSREEMTKNQVSLTCLVKGFYPSDIAVEWESNGQ |
| | PENNYKTTPPVLDSDGSFLLYSKLTVDKSRWQQGNVFSCSVMHEALH |
| | NHYTQKSLSLSPGK |
| 11 | QVQLVESGGGVVQPGRSLRLSCAASGFIFSSYAMHWVRQAPGKGLEW |
| | VAFMSYDGSNKKYADSVKGRFTISRDNSKNTLYLQMNSLRAEDTAVY |
| | YCARDRGIAAGGNYYYYGMDVWGQGTTVTVSS |
| 12 | EIVLTQSPATLSLSPGERATLSCRASQSVYSYLAWYQQKPGQAPRLLIY |
| | DASNRATGIPARFSGSGSGTDFTLTISSLEPEDFAVYYCQQRSNWPPFTF |
| | GPGTKVDIK |
| 13 | QVQLLESGGGLVQPGGSLRLSCAASGFTFSSYAMSWVRQAPGKGLEW |
| | VSTISLTSGFTYYADSVKGRFTISRDNSKNTLYLQMNSLRAEDTAVYY |
| | CARQLTLDVWGQGTLVTVSS |
| 14 | QSVLTQPPSVSVAPGQTARISCSGDNLGDKYANWYQQKPGQAPVLVI |
| | YDDIDRPSGIPERFSGSNSGNTATLTISGTQAEDEADYYCGSYDFFLGMI |
| | VFGGGTKLTVL |

The CDR sequences of the antibody mAb 9762 are as follows (Kabat definition):

(HCDR1 of TNF-α binding domain of mAb 9762)
SEQ ID NO: 15
SYAMH
(HCDR2 of TNF-α binding domain of mAb 9762)
SEQ ID NO: 16
FMSYDGSNKKYADSVKG
(HCDR3 of TNF-α binding domain of mAb 9762)
SEQ ID NO: 17
DRGIAAGGNYYYYGMDV
(LCDR1 of TNF-α binding domain of mAb 9762)
SEQ ID NO: 18
RASQSVYSYLA
(LCDR2 of TNF-α binding domain of mAb 9762)
SEQ ID NO: 19
DASNRAT
(LCDR3 of TNF-α binding domain of mAb 9762)
SEQ ID NO: 20
QQRSNWPPFT
(HCDR1 of IL-17A binding domain of mAb 9762)
SEQ ID NO: 21
SYAMS
(HCDR2 of IL-17A binding domain of mAb 9762)
SEQ ID NO: 22
TISLTSGFTYYADSVKG
(HCDR3 of IL-17A binding domain of mAb 9762)
SEQ ID NO: 23
LTLDV
(LCDR1 of IL-17A binding domain of mAb 9762)
SEQ ID NO: 24
SGDNLGDKYAN
(LCDR2 of IL-17A binding domain of mAb 9762)
SEQ ID NO: 25
DDIDRPS
(LCDR3 of IL-17A binding domain of mAb 9762)
SEQ ID NO: 26
GSYDFFLGMIV

### EXAMPLE 2

### Antigens Used in the Studies

Various formats of TNF-α and IL-17A proteins were used in the characterization of the antibodies of the invention. The proteins were expressed and purified using standard methods. The amino acid sequences of the protein used are shown below.

human IL-17A
SEQ ID NO: 3
GITIPRNPGCPNSEDKNFPRTVMVNLNIHNRNTNTNPKRSSDYYNRSTSP
WNLHRNEDPERYPSVIWEAKCRHLGCINADGNVDYHMNSVPIQQEILVLR
REPPHCPNSFRLEKILVSVGCTCVTPIVHHVA
mouse IL-17A
SEQ ID NO: 27
AAIIPQSSACPNTEAKDFLQNVKVNLKVFNSLGAKVSSRRPSDYLNRSTS
PWTLHRNEDPDRYPSVIWEAQCRHQRCVNAEGKLDHHMNSVLIQQEILVL
KREPESCPFTFRVEKMLVGVGCTCVASIVRQAA
cynomolgus IL-17A
SEQ ID NO: 28
GIAIPRNSGCPNSEDKNFPRTVMVNLNIHNRNTSTNPKRSSDYYNRSTSP
WNLHRNEDPERYPSVIWEAKCRHLGCVKADGNVDYHMNSVPIQQEILVLR
REPRHCPNSFRLEKILVSVGCTCVTPIVHHVA
rat IL-17A
SEQ ID NO: 29
AVLIPQSSVCPNAEANNFLQNVKVNLKVLNSLSSKASSRRPSDYLNRSTS
PWTLSRNEDPDRYPSVIWEAQCRHQRCVNAEGKLDHHMNSVLIQQEILVL
KREPEKCPFTFRVEKMLVGV GCTCVSSIVRHAS
rhTNF-α (recombinant human)
SEQ ID NO: 2
VRSSSRTPSDKPVAHVVANPQAEGQLQWLNRRANALLANGVELRDNQLVV
PSEGLYLIYSQVLFKGQGCPSTHVLLTHTISRIAVSYQTKVNLLSAIKSP
CQRETPEGAEAKPWYEPIYLGGVFQLEKGDRLSAEINRPDYLDFAESGQV
YFGIIAL
mouse TNF-α
SEQ ID NO: 30
MLRSSSQNSSDKPVAHVVANHQVEEQLEWLSQRANALLANGMDLKDNQLV
VPADGLYLVYSQVLFKGQGCPDYVLLTHTVSRFAISYQEKVNLLSAVKSP
CPKDTPEGAELKPWYEPIYLGGVFQLEKGDQLSAEVNLPKYLDFAESGQV
YFGVIAL
cynomolgus TNF-α
SEQ ID NO: 31
VRSSSRTPSDKPVAHVVANPQAEGQLQWLNRRANALVANGVELTDNQLVV
PSEGLYLIYSQVLFKGQGCPSNHVLLTHTISRIAVSYQTKVNLLSAIKSP
CQRETTEGAEAKPWYEPIYLGGVFQLEKGDRLSAEINLPDYLDFAESGQV
YFGIIAL
rat TNF-α
SEQ ID NO: 32
MLRSSSQNSSDKPVAHVVANHQAEEQLEWLSQRANALLANGMDLKDNQLV
VPADGLYLIYSQVLFKGQGCPDYVLLTHTVSRFAISYQEKVSLLSAIKSP
CPKDTPEGAELKPWYEPMYLGGVFQLEKGDLLSAEVNLPKYLDITESGQV
YFGVIAL

### EXAMPLE 3

### Affinity and Cross-Reactivity of the Bispecific Anti-TNF-α/IL-17A Antibodies

### Affinity to Human TNF-α and IL-17A

Biacore 3000 was used to measure the kinetic affinities of the bispecific mAbs and parental monospecific anti-TNF-α and anti-IL-17 mAbs. Short-and-long dissociation methods to enhance the estimation of affinity of tight binders with Off-rate values less than 10⁻⁵.

### Methods

The interactions of bispecific anti-TNF-α/IL-17A antibodies or parental mAbs with antigens were studied by Biacore. The experiments were performed using a Biacore 3000 and all experiments were performed in PBS (with 100 μg/mL BSA, and 0.01% P20) at 25° C. The antibodies were captured (75-200 response units) onto the sensor chip surface using an anti-IgG Fcγ antibody (16,000RU). Capture of the parental mAb or bispecific mAb was followed by injection of antigens in solution (4 serial dilutions of antigen). The association was monitored for 3 minutes in all experiments (150 μL injected at 50 μL/min). The dissociation was monitored for 20 minutes to 2 hours depending on the Off-rate. Regeneration of the sensor surface was performed with a 9 second injection of 100 mM H₃PO₄. The collected data were processed and fitted to a 1:1 Langmuir binding model. The result for each mAb was reported in the format of Ka (On-rate), Kd (Off-rate) and K_{D} (equilibrium dissociation constant).

### Materials

Sensor chip: CMS (Biacore, cat#BR-1000-14)
Goat anti human IgG Fc: Jackson cat#109-005-098
Rabbit anti-Mouse IgG Fc: Jackson cat#315-005-046
EDC, NHS, Ethanolamine Biacore, cat#BR-1000-50
10mM Sodium Acetate pH5.0: Biacore, cat#BR-1003-51
50mM NaOH: Biacore, cat#BR-1003-58
100mM Phosphoric Acid: Sigma, Cat#79617(85% concentrated=17.1M)
Dulbecco's PBS (Invitrogen product #14190)
Surfactant P20 (Biacore, Cat#BR-1000-54)
100 mM HC1 (Sigma, Cat#84428)
0.1% SDS (Sigma, Cat#L4522)
BSA (Jackson ImmunoResearch product # 001-000-161)
Buffer Preparation: Filter-sterilize and degas both buffers
Immobilization running buffer (IRB)-for coupling anti-IgG Fc: Dulbecco's PBS (D-PBS) containing 0.01% surfactant P20.
Biacore running buffer (BRB): Dulbecco's PBS (D-PBS) containing 0.01% surfactant P20, 100ug/mL BSA.

Table 6 and Table 7 show the summary of kinetics affinity data for binding to human TNF-α and human IL-17A, respectively. The parameters reported were obtained from two independent experiments using a 1:1 Langmuir binding model. Affinity, K_{D}=kd/ka. The bispecific antibodies had comparable affinities when compared to the parental mAbs. CNTO 148 is golimumab.

TABLE 6

| | Affinity to human TNF-α | | |
|---|---|---|---|
| mAb | ka (1/Ms) | kd (1/s) | K_{D} (M) |
| CNTO 148 | 1.52E+06 | 4.53E−05 | 2.98E−11 |
| 9762 | 2.02E+06 | 3.81E−05 | 1.88E−11 |
| 9762 | 1.72E+06 | 3.63E−05 | 2.11E−11 |
| 8759 | 1.75E+06 | 4.07E−05 | 2.32E−11 |
| 8759 | 1.96E+06 | 3.76E−05 | 1.92E−11 |

TABLE 7

| | Affinity to human IL-17A | | |
|---|---|---|---|
| mAb | ka (1/Ms) | kd (1/s) | K_{D} (M) |
| 6785 | 1.55E+06 | 4.79E−05 | 3.09E−11 |
| 9762 | 1.88E+06 | 4.15E−05 | 2.20E−11 |
| 9762 | 8.82E+05 | 3.99E−05 | 4.53E−11 |
| 8759 | 1.78E+06 | 4.36E−05 | 2.44E−11 |
| 8759 | 9.61E+05 | 3.74E−05 | 3.89E−11 |

### Cross-Reactivity

The generated bispecific anti-TNF-α/IL-17A antibodies were assessed for their binding to cynomolgus, mouse, and rat TNF-α and IL-17A. The experimental procedure was similar to measuring affinity to human antigens.

Table 8 and Table 9 show the kinetics affinity data for binding to TNF-α and IL-17A from various species, respectively. The parameters reported were obtained from two independent experiments. While mAb 6785 (original parental anti-IL-17A mAb) and the bispecific mAbs 9762 and 8759 bound to mouse and rat IL-17A, there was minimal neutralization in these species.

TABLE 8

| Antigen | mAb | ka (1/Ms) | kd (1/s) | K_{D} (M) |
|---|---|---|---|---|
| Cyno TNF-α | CNTO 148 | 2.11E+05 | 1.44E−04 | 6.81E−10 |
| | 9762 | 2.59E+05 | 1.03E−04 | 3.99E−10 |
| | 9762 | 6.81E+05 | 1.27E−04 | 1.86E−10 |
| | 8759 | 2.59E+05 | 1.14E−04 | 4.40E−10 |
| | 8759 | 7.10E+05 | 1.14E−04 | 1.60E−10 |
| Mouse TNF-α | 9762 | — | — | No Binding |
| | 8759 | — | — | No Binding |
| Rat TNF-α | 9762 | — | — | No Binding |
| | 8759 | — | — | No Binding |

The parameters reported were obtained from a 1:1 Langmuir binding model.
Affinity, K_{D} = kd/ka.
No binding was observed for mouse and rat TNF-α at concentrations up to100 nM.

TABLE 9

| Antigen | mAb | ka (1/Ms) | kd (1/s) | K_{D} (M) |
|---|---|---|---|---|
| Cyno IL-17A | 6785 | 1.29E+06 | 3.68E−05 | 2.86E−11 |
| | 9762 | 1.55E+06 | 3.66E−05 | 2.36E−11 |
| | 9762 | 1.43E+06 | 4.57E−05 | 3.18E−11 |
| | 8759 | 1.35E+06 | 3.59E−05 | 2.65E−11 |
| | 8759 | 1.59E+06 | 4.40E−05 | 2.77E−11 |
| Mouse IL-17A | 9762 | 1.03E+06 | 1.03E−03 | 9.97E−10 |
| | 8759 | 9.99E+05 | 1.01E−03 | 1.01E−09 |
| Rat IL-17A | 9762 | 2.05E+05 | 3.16E−03 | 1.54E−08 |
| | 8759 | 1.97E+05 | 2.74E−03 | 1.39E−08 |

The parameters reported were obtained from a 1:1 Langmuir binding model.
Affinity, K_{D} = kd/ka

### EXAMPLE 4

### The Bispecific Anti-TNF-α/IL-17A Antibodies Inhibit TNF-α-Dependent Functions

The bispecific antibodies mAb 9762 and mAb 8759 were tested for their ability to inhibit TNF-α binding to its receptor and to inhibit soluble or membrane-bound recombinant or endogenous TNF-α-mediated cytotoxicity. The parental antibodies mAb 9809, mAb 6696, mAb 4782 and mAb 7206 as well as ENBREL® (etanercept) and SIMPONI® (golimumab) were used as controls in the studies.

### Inhibition of Soluble Recombinant Human TNF-α Mediated Cytotoxicity on WEHI-164 Cells

Neutralizing potency of the bispecific anti-TNF-α/IL-17A antibodies were measured in a recombinant human (rhTNF-α) (SEQ ID NO: 2) -induced cytotoxicity assay in WEHI-164 mouse fibrosarcoma cell line expressing endogenous mouse TNF-α receptors. WEHI-164 mouse fibrosarcoma cell line was obtained from Dr. Marc Feldmann (Kennedy Institute, London, UK) (Espevik and Nissen-Meyer J Immunol Methods. 1986 Dec 4;95(1):99-105). Serial dilutions of each mAb were incubated with 0.1 ng/mL of recombinant human (rh) TNF-α followed by overnight incubation with 5×10⁴ cells/well of WEHI-164 cells. Cell viability was measured by the Celltiter Glo method. The experiment was repeated three times. Table 10 shows the results expressed as mean IC₅₀ with 95% confidence interval for three individual experiments. mAb 9762 and mAb 8759 neutralized the cytotoxic effect of rhTNF-α in a concentration-dependent manner with ˜3 fold higher of IC₅₀ than the parental anti-TNF-α antibodies mAb 9809 and mAb 6696. Etanercept (ENBREL®) and CNTO148 (golimumab) performed as expected in this assay. No inhibition was observed with anti-IL-17A parental antibodies mAb 4782 and mAb 7206.

TABLE 10

| | IC₅₀ (nM); | | |
|---|---|---|---|
| | WEHI-164 cells cytotoxicity mediated by soluble rhTNF-α | | |
| mAb | Experiment 1 | Experiment 2 | Experiment 3 |
| 9762 | 1.277 | 2.071 | 0.6307 |
| | (1.183 to 1.378) | (1.863 to 2.302) | (0.5561 to 0.7152) |
| 8759 | 1.2 | 1.734 | 0.4779 |
| | (1.109 to 1.298) | (1.570 to 1.916) | (0.4192 to 0.5448) |
| 9809 | 0.586 | 0.9897 | 0.2986 |
| | (0.548 to 0.627) | (0.8920 to 1.098) | (0.2539 to 0.3512) |
| 6696 | 0.380 | 0.8252 | 0.2176 |
| | (0.331 to 0.436) | (0.7131 to 0.9548) | (0.1801 to 0.2628) |

95% confidence interval shown in the parenthesis in the table

Inhibition of Soluble Human Recombinant TNF-α Mediated Cytotoxicity in KYM cClls

Neutralizing potency of the bispecific anti-TNF-α/IL-17A antibodies were measured in a recombinant human (rhTNF-α)-induced cytotoxicity assay in KYM-1D4 human rhabdomyosarcoma cell line endogenously expressing human TNF-α receptors. KYM-1D4 cell line was obtained from Marc Feldmann (Kennedy Institute, London, UK; Butler et al., (1994) Cytokine 6:616-23). KYM-1D4 cells were seeded into 96-well microtiter plates (5×10⁴ cells in 50 μL/well) and incubated 4 hours at 37° C. Serial dilutions of each mAb were pre-incubated with 100 pg/ml of rhTNF-α (SEQ ID NO: 2) (the concentration of rhTNF-α was confirmed to induce more than 85% of KYM-1D4 cells death in each assay) in medium containing 1 μg/mL as the final concentration of actinomycin D followed by overnight incubation with the cells. Cell viability was read by Celltiter Glo method. The experiment was repeated three times, and the results were expressed as mean IC₅₀ with 95% confidence interval for each experiment. The bispecific antibodies mAb 9762 and mAb 8759 neutralized the cytotoxic effect of rhTNF-α in a concentration-dependent manner, with ˜3 fold higher IC₅₀ than the parental anti-TNF-α antibodies mAb 9809 and mAb 6696 (Table 11). Etanercept (ENBREL®) and CNTO 148 (golimumab) performed as expected in this assay. No inhibition was observed with the parental anti-IL-17A antibodies mAb 4782 and mAb 7206.

TABLE 11

| | IC₅₀ (nM); | | |
|---|---|---|---|
| | KYM-1D4 cells, cytotoxicity mediated by soluble rhTNF-α | | |
| mAb | Experiment 1 | Experiment 2 | Experiment 3 |
| 9762 | 0.1519 | 0.3429 | 0.1687 |
| | (0.0981 to 0.2353) | (0.2799 to 0.4202) | (0.1305 to 0.2181) |
| 8759 | 0.1295 | 0.2607 | 0.1473 |
| | (0.09229 to 0.1818) | (0.2251 to 0.3019) | (0.1220 to 0.1778) |
| 9809 | 0.06494 | 0.09555 | 0.06764 |
| | (0.05096 to 0.08275) | (0.08106 to 0.1126) | (0.05607 to 0.08160) |
| 6696 | 0.04633 | 0.08055 | 0.04987 |
| | (0.03539 to 0.06065) | (0.06748 to 0.09615) | (0.04166 to 0.05970) |

95% confidence interval shown in the parenthesis in the table

### Inhibition of Human Transmembrane TNF-α Mediated Cytotoxicity

To investigate whether the bispecific anti-TNF-α/IL-17A antibodies were able to neutralize human transmembrane TNF-α, cytotoxicity induced by protease-resistant form of transmembrane TNF-α overexpressed by K2 cells (a rhTNF-α stably transfected mouse myeloma cell line) was measured using the cytotoxicity assay described above for the KYM-1D4 rhabdomyosarcoma cell line. K2 cells were prepared by transfecting murine SP2/0 myeloma cells with the plasmid encoding a mutant form of human TNF-αthat lacks amino acids Val l to Pro12. TNF-α with this deletion has been shown to be resistant to ADAM17-mediated proteolytic cleavage that releases mature, soluble TNF from the cell surface (Perez et al., Cell. 1990; 63:251-258). Concentration-dependent neutralization was seen with both bispecific anti-TNF-α/IL-17A antibodies mAb 9762 and mAb 8759, and the parental anti-TNF-α antibodies mAb 9809 and mAb 6696. No neutralization was observed with the parental anti-IL-17A antibodies mAb 4782 and mAb 7206. The IC₅₀ values were within ˜4 fold higher IC₅₀ for the bispecific mAbs when compared to the parental antibodies (Table 12).

TABLE 12

| | IC₅₀ (nM); | | |
|---|---|---|---|
| | KYM-1D4 cells, cytotoxicity mediated by | | |
| | membrane bound human TNF-α | | |
| mAb | Experiment 1 | Experiment 2 | Experiment 3 |
| 9762 | 0.8196 | 0.966 | 0.4249 |
| | (0.6705 to 1.002) | (0.8162 to 1.145) | (0.3607 to 0.5004) |
| 8759 | 0.6532 | 0.8828 | 0.3002 |
| | (0.5604 0.7613) | (0.7446 to 1.047) | (0.2661 to 0.3387) |
| 9809 | 0.2123 | 0.3528 | 0.1941 |
| | (0.1807 to 0.2495) | (0.2730 to 0.4557) | (0.1642 to 0.2294) |
| 6696 | 0.2082 | 0.2348 | 0.1405 |
| | (0.1862 to 0.2327) | (0.1951 to 0.2826) | (0.1189 to 0.1660) |

95% confidence interval shown in the parenthesis in the table

### Inhibition of Human Endogenous Soluble TNF-α Mediated Vytotoxicity on KYM Cells

LPS-stimulated human monocytes were used as a source of native or natural human TNF-α to compare the neutralization capacity of the bispecific anti-TNF-α/IL-17A antibodies mAb 9762 and mAb 8759 and the parental anti-TNF-α antibodies mAb 9809 and mAb 6696.

The ability of the bispecific anti-TNF-α/IL-17A mAbs to neutralize native human endogenous TNF-α secreted by primary monocytes was assessed in cytotoxicity assays using KYM-1D4 cells as described above. The bispecific anti-TNF-α/IL-17A mAbs 9762 and mAb 8759 neutralized the cytotoxic effect of endogenous TNF-α in a concentration-dependent manner with ˜3 fold higher IC₅₀ than the parental anti-TNF-α antibodies. No neutralization was observed with parental anti-IL-17A antibodies mAb 4782 and mAb 7206. Table 13 shows the IC₅₀ values obtained in this assay.

TABLE 13

| | IC₅₀ (nM); | | |
|---|---|---|---|
| | KYM-1D4 cells, cytotoxicity mediated by endogenous | | |
| | soluble TNF-α | | |
| mAb | Experiment 1 | Experiment 2 | Experiment 3 |
| 9762 | 0.3463 | 0.3269 | 0.1965 |
| | (0.3153 to 0.3804) | (0.3080 to 0.3470) | (0.1833 to 0.2108) |
| 8759 | 0.2834 | 0.2943 | 0.1432 |
| | (0.2701 to 0.2975) | (0.2711 to 0.3196) | (0.1334 to 0.1537) |
| 9809 | 0.1358 | 0.1415 | 0.06381 |
| | (0.1259 to 0.1465) | (0.1305 to 0.1534) | (0.05613 to 0.07254) |
| 6696 | 0.1033 | 0.09212 | 0.06378 |
| | (0.09269 to 0.1152) | (0.08251 to 0.1028) | (0.05603 to 0.07260) |

95% confidence interval shown in the parenthesis in the table

### Neutralization of TNF-α From Different Animal Species

The ability of the bispecific anti-TNF-α/IL-17A antibodies to neutralize mouse, rat and cynomolgus monkey recombinant TNF-α was assessed in cytotoxicity assays using WEHI -164 cells as described above. The bispecific mAbs, similarly to CNTO 148 (golimumab) and the parental anti-TNF-α antibodies did not inhibit mouse or rat TNF-α, while they inhibited cynomolgus TNF-α with potency within ˜3 fold weaker compared to the parental anti-TNF-α antibodies. No neutralization was observed with parental anti-IL-17A antibodies mAb 4782 and mAb 7206. In concordance with affinity binding measurement, neutralization of cynomolgus TNF-α was weaker compared to human recombinant TNF-α by the bispecific antibodies. Table 14 shows the ICso values for cyno TNF-α inhibition.

TABLE 14

| | IC₅₀ (nM); | | |
|---|---|---|---|
| | Inhibition of cyno TNF-α mediated cytotoxicity in | | |
| | WEHI-164 cells | | |
| mAb | Experiment 1 | Experiment 2 | Experiment 3 |
| 9762 | 4.418 | 20.64 | 9.294 |
| | (3.257 to 5.992) | (18.12 to 23.51) | (4.177 to 20.68) |
| 8759 | 3.816 | 14.93 | 7.324 |
| | (3.051 to 4.774) | (13.11 to 17.00) | (4.820 to 11.13) |
| 9809 | 2.207 | 5.998 | 4.976 |
| | (1.713 to 2.845) | (5.287 to 6.805) | (2.567 to 9.645) |
| 6696 | 1.459 | 4.79 | 2.468 |
| | (0.7023 to 3.033) | (4.329 to 5.300) | (1.772 to 3.436) |

95% confidence interval shown in the parenthesis in the table

### EXAMPLE 5

### The Bispecific Anti-TNF-α/IL-17A Antibodies Inhibit IL-17A-Dependent Functions

### Neutralization of Recombinant Human IL-17A Binding to IL-17RA Receptor

The bispecific anti-TNF-α/IL-17A antibodies mAb 9762 and mAb 8759 inhibited the binding of biotinylated human recombinant IL-17A to IL-17RA receptor in a concentration-dependent manner with ˜3 fold higher IC₅₀ than the parental anti-IL-17A parental antibodies mAb 4782 and mAb 7206. No inhibition was observed with an isotype control antibody.

For the assay, clear maxisorp plates were coated with soluble recombinant human IL-17RA-Fc chimeric protein (rhIL-17R-Fc, R&D Systems, catalogue #177-IR, 0.25 μg/well) in 0.1 M sodium carbonate-bicarbonate buffer, pH 9.4 and incubated overnight at 4° C. The plates were blocked for 1 hour with ELISA block buffer (1% BSA, 5% Sucrose and 0.05% Sodium Azide in PBS) and washed three times with wash buffer (0.05% Tween-20 in PBS). After washing, 25 ng/mL of biotinylated rhIL-17A was pre-incubated for 5-10 minutes with a dilution series (30-0.0015 μg/mL) of mAbs or irrelevant IgG1 isotype control antibody mAb 1787. After pre-incubation of rhIL-17A and mAbs, the mixture was added to IL-17RA-coated plates. Plates were washed three times with wash buffer, and then incubated with SA-HRP (Jackson Immunoresearch) for 20 minutes at RT. Plates were washed three times with ELISA wash buffer. Following the wash, TMB substrate or OPD (BD & Sigma respectively) was added to each well and incubated until the appropriate color change was detected. The reaction was stopped with the addition of 2N sulfuric acid. Colorimetric intensity was then determined by reading the plate at a wavelength of 450 or 492 nM (TMB & OPD respectively) using a spectrophotometer (SpectramaxPlus, Molecular Devices). ICso values were determined by non-linear regression using GraphPad Prism software (GraphPad Software, Inc). The results were plotted as mean values. Table 15 shows the mean ICso values with the 95% confidence interval values of inhibition in the parenthesis.

TABLE 15

| | IC₅₀ (μg/mL); | | |
|---|---|---|---|
| | Inhibition IL-17A/ IL-17RA interaction | | |
| Ab | Experiment 1 | Experiment 2 | Experiment 3 |
| 9762 | 0.1041 | 0.1682 | 0.2590 |
| | (0.09343-0.1160) | (0.1474-0.1920) | (0.2433-0.2757) |
| 8759 | 0.1270 | 0.1743 | 0.2538 |
| | (0.1171-0.1378) | (0.1639-0.1853) | (0.2415-0.2667) |
| 4782 | 0.04028 | 0.04264 | 0.07601 |
| | (0.03759-0.04316) | (0.04003-0.04542) | (0.07179-0.08047) |
| 7206 | 0.04095 | 0.03067 | 0.07167 |
| | (0.03924-0.04274) | (0.02829-0.03326) | (0.06727-0.07636) |

95% Confidence Interval Values of Inhibition shown in parenthesis in the table

### Neutralization of Recombinant and Native Human IL-17A Induced IL-6 and GROα Production From Human Dermal Fibroblasts

The effect of the bispecific anti-TNF-α/IL-17A antibodies on IL-17A-induced cytokine production was examined using a cell-based bioassay. Recombinant human IL-17A in the presence of rhTNF-α stimulated primary normal human dermal fibroblasts (NHDFs) to produce multiple cytokines, including GROα and IL-6.

Brielfy, Normal Human Dermal Fibroblasts (NHDF, Lonza) cells were seeded into a 96-well flat bottom tissue culture plate at 5,000 cells per well in FGM-2 medium (Lonza) and incubated overnight (37° at 5% CO₂). Following incubation, 10 ng/mL rhTNF-α and rhlL-17A at 10 ng/mL was pre-incubated with a dilution series (30-0.0015 μg/mL) of mAbs 9762, 8759, 4782, 7206 or irrelevant IgG1 isotype control antibody 1787 and the mixture was then added to NHDF cells. IL-17A and TNF-α samples with no antibody added were included as controls, while samples consisting of culture medium only were included as negative controls. Cells were incubated for 24 h (37°, 5% CO₂) and culture supernatants were collected and assayed by ELISA for IL-6 and GROα using human Duo Sets (R&D Systems, Inc.). IC₅₀ values were determined by non-linear regression using GraphPad Prism software (GraphPad Software, Inc). The results were shown as mean values.

The bispecific antibodies mAb 9762 and mAb 8759 inhibited GROα (Table 16) and IL-6 (Table 17) production in a concentration-dependent manner with ˜4 to 5 fold higher ICso than the parental anti-IL-17A antibodies mAb 4782 and mAb 7206. No inhibition was observed with an isotype control antibody, CNTO 1787. As a low concentration of TNF-α was added to the culture to amplify IL-17A mediated cytokine secretion, the parental anti-TNF-α antibodies mAb 9809 and mAb 6696 partially inhibited GROα and IL-6 production.

The bispecific anti-TNF-α/IL-17A antibodies were also assessed for their ability to block native IL-17A. Briefly, CD4⁺ T cells were isolated and polarized to a Tₕ17 phenotype for 5 days. The supernatant was harvested and human IL-17 was purified using affinity purification isolation methods. Normal human dermal fibroblast cells were stimulated with 0.5% native IL-17A supernatant in the presence of recombinant human TNF-α (0.1 ng/mL) and GROα secretion was assessed as described previously. The bispecific anti-TNF-α/IL-17A antibodies mAb 9762 and mAb 8759 as well as the parental anti-IL-17A antibody mAb 4782 inhibited human native IL-17A induced IL-6 and GROα release in a dose-dependent manner These data demonstrated the bispecific anti-TNF-α/IL-17A antibodies mAb 9762 and mAb 8759 neutralized native human IL-17A protein (data not shown).

TABLE 16

| | IC₅₀ (μg/mL); | | |
|---|---|---|---|
| | Inhibition of native IL-17A mediated GROα | | |
| | production by NHDF | | |
| mAb | Experiment 1 | Experiment 2 | Experiment 3 |
| 9762 | 0.08099 | 0.06478 | 0.05081 |
| | (0.07591-0.08642) | (0.04602-0.09120) | (0.04163-0.06201) |
| 8759 | 0.07548 | 0.06921 | 0.06526 |
| | (0.0698-0.08163) | (0.05197-0.09216) | (0.05903-0.07215) |
| 4782 | 0.02073 | 0.03274 | 0.02362 |
| | (0.01939-0.02216) | (0.02272-0.04717 | (0.01905-0.02929) |
| 7206 | 0.01977 | 0.02646 | 0.02148 |
| | (0.0162-0.02413) | (0.02259-0.03100) | (0.02089-0.02210) |

95% Confidence Interval Values of Inhibition shown in parenthesis in the table
NHDF: Normal human dermal fibroblast

TABLE 17

| | IC₅₀ (μg/mL); | | |
|---|---|---|---|
| | Inhibition of native IL-17A mediated IL-6 production by NHDF | | |
| mAb | Experiment 1 | Experiment 2 | Experiment 3 |
| 9762 | 0.05009 | 0.04450 | 0.04895 |
| | (0.04673-0.05369) | (0.03855-0.05137) | (0.03909-0.06131) |
| 8759 | 0.04775 | 0.03799 | 0.06083 |
| | (0.04254-0.05360) | (0.02278-0.06335) | (0.04698-0.07877) |
| 4782 | 0.02542 | 0.03357 | 0.02420 |
| | (0.02091-0.03090) | (0.00981-0.1149 | (0.02057-0.02847) |
| 7206 | 0.02142 | 0.02423 | 0.02318 |
| | (0.01987-0.02308) | (0.01869-0.03141) | (0.02078-0.02584) |

95% Confidence Interval Values of Inhibition shown in parenthesis in the table
NHDF: Normal human dermal fibroblast

### Neutralization of Recombinant Human IL-17A/F Heterodimer Induced IL-6 and GROα Production From Normal Human Dermal Fibroblasts

IL-17A can pair with IL-17F to form a heterodimeric IL-17A/F cytokine, which has similar biological activities as the IL-17A homodimer. The neutralization potency of the bispecific antibodies in inhibiting IL-17A/F-induced cytokine production was examined using a cell-based bioassay described above. mAbs 9762 and 8759 inhibited GROα (Table 18) and IL-6 (Table 19) production from primary normal human dermal fibroblasts (NHDFs) in a concentration-dependent manner with comparable IC₅₀ relative to the parental anti-IL-17 antibodies mAb 4782 and mAb 7206. No inhibition was observed with an isotype control antibody, mAb 1787. As a low concentration of TNF-α was added to the culture to amplify IL-17A/F mediated cytokine secretion, the anti-TNF-α parental antibodies mAb 9809 and mAb 6696 also inhibited GROα and IL-6 production.

TABLE 18

| | IC₅₀ (μg/mL); | | |
|---|---|---|---|
| | Inhibition of IL-17A/F mediated GROα production by NHDF | | |
| mAb | Experiment 1 | Experiment 2 | Experiment 3 |
| 9762 | 0.5490 | 0.4359 | 0.3920 |
| | (0.2118-1.423) | (0.1810-1.050) | (0.2566-0.5988) |
| 8759 | 0.2827 | 0.4601 | 0.4346 |
| | (0.1256-0.6364) | (0.1968-1.075) | (0.2550-0.7409) |
| 4782 | 0.4726 | 0.1165 | 0.3167 |
| | (0.1525-1.465) | (0.01359-0.9989 | (0.2476-0.4050) |
| 7206 | 0.7245 | 0.3925 | 0.4030 |
| | (0.1925-2.726) | (0.2251-0.6843) | (0.3155-0.5147) |

95% Confidence Interval Values of Inhibition shown in parenthesis in the table
NHDF: Normal human dermal fibroblast

TABLE 19

| | IC₅₀ (μg/mL); | | |
|---|---|---|---|
| | Inhibition of IL-17A/F mediated IL-6 production by NHDF | | |
| mAb | Experiment 1 | Experiment 2 | Experiment 3 |
| 9762 | 0.1349 | 0.1742 | 0.1305 |
| | (0.06112-0.2978) | (0.1052-0.2887) | (0.08833-0.1927) |
| 8759 | 0.1170 | 0.1330 | 0.1023 |
| | (0.05619-0.2435) | (0.09237-0.1914) | (0.06222-0.1683) |
| 4782 | 0.7138 | 0.2427 | 0.9898 |
| | (0.3399-1.499) | (0.05881-1.002 | (0.5486-1.786) |
| 7206 | 0.3437 | 0.2688 | 0.5452 |
| | (0.1262-0.9361) | (0.06188-1.167) | (0.2603-1.142) |

95% Confidence Interval Values of Inhibition shown in parenthesis in the table
NHDF: Normal human dermal fibroblast

### Cross-Reactivity of mAb 9762 With Human Recombinant IL-17F

IL-17F is the closest related cytokine in the IL-17 family, sharing 50% identity at the amino acid level to IL-17A. To determine whether the bispecific anti-TNF-α/IL-17A antibodies were specific for IL-17A, binding of mAbs 9762 and 8759 to rhIL-17A was assessed in the absence or presence of IL-17F. Both bispecific mAbs were able to bind IL-17A in the presence of recombinant human IL-17F, similar to the parental anti-IL-17A antibodies, suggesting that the mAbs 9762 and 8759 were specific for IL-17A and did not cross-react with IL-17F. In concordance with these results, mAbs 9762 and 8759 inhibited TNF-α mediated but not IL-17F mediated GROα secretion in TNF-α amplified IL-17F-induced cytokine release assay from primary human dermal fibroblasts (data not shown).

### Cross-Reactivity of Bispecific Anti-TNF-α/IL-17A Antibodies With Recombinant Mouse, Rat and Cynomolgus IL-17A

The bispecific anti-TNF-α/IL-17A antibodies mAb 9762 and mAb 8759 did not inhibit recombinant rat or mouse IL-17A induced KC secretion from mouse embryonic fibroblast NIH3T3 cell line, similarly to the parental anti-IL-17A parental antibodies mAb 4782 and mAb 7206 (data not shown).

The bispecific anti-TNF-α/IL-17A antibodies mAb 9762 and mAb 8759 as well as the parental anti-IL-7A antibodies mAb 4782 and mAb 7206 inhibited recombinant cynomolgus IL-17A induced IL-6 and GROα secretion from Normal Human Dermal Fibroblasts.

Normal Human Dermal Fibroblasts (NHDF, Lonza) cells were seeded into a 96-well flat bottom tissue culture plate at 5,000 cells per well in FGM-2 medium (Lonza) and incubated overnight (37°, 5% CO₂). Following incubation, 10 ng/mL recombinant cyno TNF-α and recombinant cyno IL-17A at 10 ng/mL was pre-incubated with a dilution series 30-0.0015 μg/mL of mAbs 9762, 8759, 4782, 7206 or irrelevant IgG1 isotype control antibody mAb1787 and the mixture was then added to NHDF cells. IL-17A and TNF-α samples with no antibody added were included as controls, while samples consisting of culture medium only were included as negative controls. Cells were incubated for 24 h (37°, 5% CO2) and culture supernatants were collected and assayed by ELISA for IL-6 and GROα using human Duo Sets (R&D Systems, Inc.). ICso values were determined by non-linear regression using GraphPad Prism software (GraphPad Software, Inc). The results were expressed as mean IC₅₀ values. Table 20 shows the IC₅₀ values of inhibition of cyno recombinant IL-17A induced GROα production from NHDFs. Table 21 shows the IC₅₀ values of inhibition of cyno recombinant IL-17A induced IL-6 production from NHDFs.

TABLE 20

| | IC₅₀ (μg/mL); | | |
|---|---|---|---|
| | Inhibition of cyno IL-17A mediated GROα production by NHDF | | |
| mAb | Experiment 1 | Experiment 2 | Experiment 3 |
| 9762 | 0.2988 | 0.3293 | 0.1368 |
| | (0.1927-0.4633) | (0.2436-0.4452) | (0.1263-0.1481) |
| 8759 | NA | NA | 0.1417 |
| | | | (0.1162-0.1728) |
| 4782 | 0.02352 | 0.04660 | 0.03488 |
| | (0.01624-0.03405) | (0.03688-0.05889 | (0.02864-0.04247) |
| 7206 | NA | NA | 0.02712 |
| | | | (0.02245-0.03275) |

95% Confidence Interval Values of Inhibition shown in parenthesis in the table
NHDF: Normal human dermal fibroblast

TABLE 21

| | IC₅₀ (μg/mL); | | |
|---|---|---|---|
| | Inhibition of cyno IL-17A mediated IL-6 production by NHDF | | |
| mAb | Experiment 1 | Experiment 2 | Experiment 3 |
| 9762 | NA | 0.07837 | 0.05827 |
| | | (0.0556-0.1105) | (0.04665-0.07280 |
| 8759 | NA | NA | 0.04758 |
| | | | (0.04260-0.05314) |
| 4782 | NA | 0.04372 | 0.02483 |
| | | (0.02835-0.06742) | (0.02123-0.02904) |
| 7206 | NA | NA | 0.03255 |
| | | | (0.02497-0.04244) |

95% Confidence Interval Values of Inhibition shown in parenthesis in the table
*NHDF: Normal human dermal fibroblast

### EXAMPLE 6

### Characterization of Functional Activity of Bispecific Anti-TNF-α/IL-17A Antibodies in TNF-α/IL-17A Dual Dependent Assays With Human Synovial Fibroblasts Isolated From RA Patients (RA-FLS)

### Neutralization of Inflammatory Responses Mediated by Human Recombinant TNF-α and IL-17A Induced in RA-FLS

Given the importance of synovial fibroblast in propagating local inflammation in RA joints, the combinatorial effects of TNF-α and IL-17A on synovial fibroblasts isolated from RA patients was investigated.

Briefly, synoviocytes isolated from RA patients were treated for 24 hours in a grid-like pattern with a dose range of 0-100 ng/mL of IL-17A alone, TNF-α alone, or the combination of IL-17A and TNF-α. The amount of IL-6, GROα and MMP-3 release into the cell supernatant was quantitated by luminexbead-based analysis. The experiment was repeated three times using three independent donors.

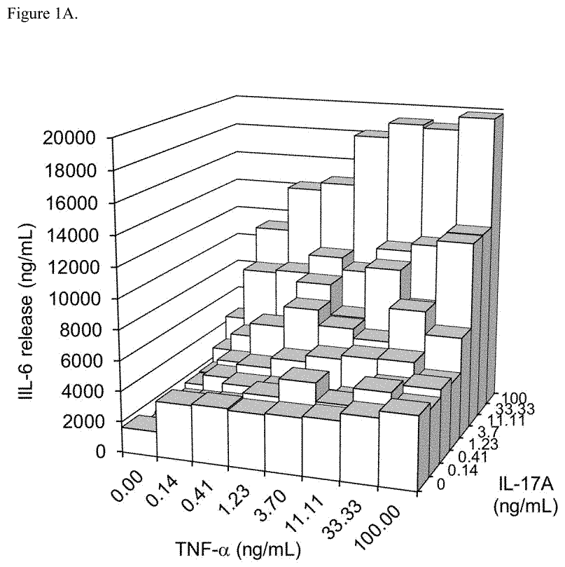

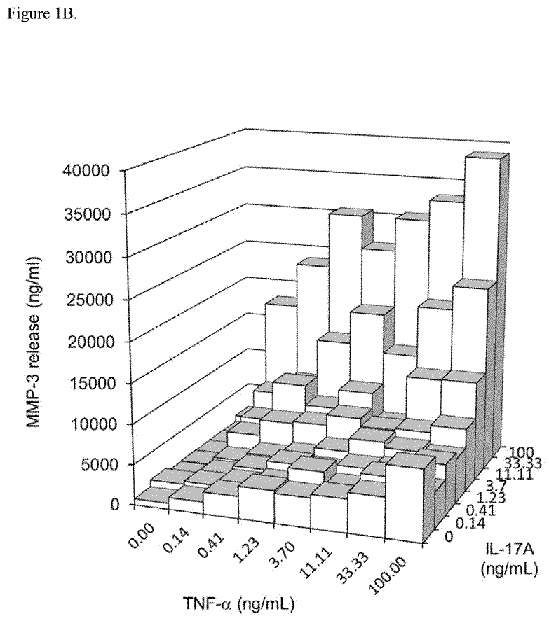

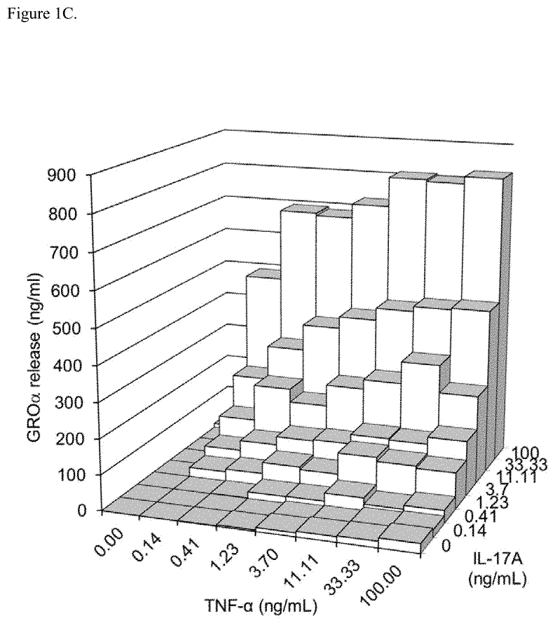

Treatment with the combination of TNF-α and IL-17A resulted in enhanced production of 12 out of 13 measured pro-inflammatory mediators. IL-6, MMP3 and GROα secretion were selected as representative data to demonstrate TNF-α/IL-17A cooperativity to induce proinflammatory mediators and tissue degradation markers, respectively. It appeared that while TNF-α alone was a more potent inducer of cytokine production relative to IL-17A, the latter was a driver of cooperativity in inducing anti-inflammatory cytokine production. Figure lA shows the amount of IL-6 released, FIG. 1B shows the amount of MMP-3 released, and FIG. 1C shows the amount of GROα released by the synovial fibroblasts upon various treatments in the vertical axis in pg/ml. Each value was calculated as the mean of triplicate wells.

Mathematical modeling of the data was generated to assess individual contributions of TNF-α or IL-17A and their interactions in induction of inflammatory responses in RA-FLS. Based on the three-dimensional modeling of the data, a mathematical equation was derived that described the shape of the data, and mathematically calculated the contributions of TNF-α and IL-17A versus the concentrations of individual cytokines released from the cells. These equations identified whether the contribution of TNF-α or IL-17A to a particular cytokine response was linear and/or exponential, indicative of how much TNF-α or IL-17A contributed to the response. In this equation, a positive exponential term indicated a higher contribution than a linear term. Hence, if TNF-α had a positive exponential term, but IL-17A did not, then TNF-α contributed more substantially to the observed response. The equation also modeled the potential interaction between TNF-α and IL-17A. If this “TNF-α:IL-17A” term was positive, then the mathematical model illustrated a synergistic response between IL-17A and TNF-α. The equation in describing IL-6 secretion from the cells was: IL-6=822(log(TNF-α))+1480(log(IL-17A))−208(log(TNF-α))2+1549(log(IL-17A))2+390(log(TNF-α)×log(IL-17A)).

The mathematical modeling confirmed synergistic responses of TNF-α and IL-17A that were mostly apparent at high concentration of IL-17A. Both TNF-α and IL-17A demonstrated positive linear terms, indicating both were responsible for cytokine release. Additionally, IL-17A had an exponential term (1549(logIL17))2, indicating an exponential increase in IL-6 as the IL-17A concentration increased. The contribution of the combination of TNF-α and IL-17A was also positive in this equation, indicating that the contribution from the combination was greater than either cytokine alone.

The bispecific anti-TNF-α/IL-17A antibodies as well as the parental monospecific TNF-α and IL-17A antibodies were evaluated for their neutralization potency of TNF-α and IL-17A-mediated cytokine release (IL-6, IL-8, RANTES, GROα, MMP-3, MCP-1, and ENA-78) in RA-FLS cells.

Briefly, FLS cells were treated overnight with a cocktail containing TNF-α and IL-17A, plus either the parental anti-TNF-α antibody (mAb 9809), the parental anti-IL-17A antibody (mAb 4782), or the bispecific antibodies mAb 9762 and mAb 8759. Antibodies and cytokines were pre-incubated for one hour before addition to cells. After overnight incubation at 37° C., the supernatant was harvested from the FLS cells and tested by luminex analysis for cytokines known to be induced by the TNF-α and IL-17A cytokine combination. IC₅₀ and confidence intervals were generated using PRISM v.6.02 based on a sigmoidal dose response.

TNF-α and IL-17A had opposing activities on RANTES release from the RA-FLS cultures, with TNF-α increasing RANTES release and IL-17A decreasing RANTES release into the cell supernatant. Thus, the parental anti-IL-17A antibody alone was incapable of inhibiting RANTES release, while the parental anti-TNF-α antibody and both bispecific antibodies mAb 9762 and mAb 8759 inhibited RANTES release down to the baseline levels. Both bispecific antibodies mAb 9762 and mAb 8759 inhibited TNF-α and IL-17A mediated cytokine release at or close to baseline (vehicle-treated controls) for all other cytokines tested. The IC₅₀ values for inhibition of IL-6 and GROα release are shown in Table 22 and Table 23, respectively. The IC₅₀ for the parental anti-TNF-α (mAb 9809) was significantly higher (p<0.05) than the IC_{50′} for the parental anti-IL-17A (mAb 4782) alone or the bispecific mAbs for inhibition of both IL-6 and GROα release.

TABLE 22

| | IC₅₀ (nM); | | |
|---|---|---|---|
| | Inhibition of rhTNF-α and IL-17 induced IL-6 secretion | | |
| | from RA-FLSs | | |
| | Donor 1 | Donor 2 | Donor 3 |
| mAb | (ST1320) | (ST1349) | (ST1359) |
| 9809 | 0.4 | 0.55 | 0.59 |
| | [0.1454 to 1.098] | [0.2217 to 1.362] | [0.1869 to 1.849] |
| 4782 | 0.34 | 0.22 | 0.25 |
| | [0.1580 to 0.7299] | [0.1252 to 0.3782] | [0.1565 to 0.4034] |
| 9762 | 0.22 | 0.18 | 0.18 |
| | [0.1224 to 0.3956] | [0.1362 to 0.2505] | [0.1087 to 0.2933] |
| 8759 | 0.23 | 0.2 | 0.26 |
| | [0.1150 to 0.4638] | [0.1524 to 0.2616] | [0.1537 to 0.4282] |

95% Confidence Interval Values in parenthesis in the table

TABLE 23

| | IC₅₀ (nM); | | |
|---|---|---|---|
| | Inhibition of rhTNF-α and IL-17A induced GROα | | |
| | secretion from RA-FLSs from three donors | | |
| | Donor 1 | Donor 2 | Donor 3 |
| mAb | (ST1320) | (ST1349) | (ST1359) |
| 9809 | 1 | 0.57 | 0.57 |
| | [0.3706 to 2.693] | [0.3031 to 1.087] | [0.2311 to 1.406] |
| 4782 | 0.25 | 0.12 | 0.14 |
| | [0.1361 to 0.4596] | [0.06960 to 0.2062] | [0.08586 to 0.2207] |
| 9762 | 0.2 | 0.12 | 0.12 |
| | [0.1261 to 0.3210] | [0.09111 to 0.1575] | [0.08426 to 0.1771] |
| 8759 | 0.22 | 0.11 | 0.17 |
| | [0.1154 to 0.4050] | [0.06779 to 0.1650] | [0.1017 to 0.2814] |

95% Confidence Interval Values in parenthesis in the table

### Neutralization of Inflammatory Responses Mediated by Human Recombinant TNF-α and IL-17A Induced in Chondrocyte-Synoviocyte Coculture

A human rheumatoid arthritis synoviocyte (FLS)-chondrocyte co-culture system was established to evaluate the activity of the bispecific anti-TNF-α/IL-17A antibodies on TNF-α and IL-17A induced responses. This co-culture system incorporated the two principal cell types found in the articular joint shown to interact with each other, modulating their respective behavior in diseased conditions.

In the studies, the co-cultures were treated with TNF-α and/or IL-17A and the secretion of cytokines was evaluated by enzyme-linked immunosorbent assay (ELISA). In addition, changes in inflammatory gene expression were evaluated by RT-PCR array analysis so to identify additional synergistically regulated genes.

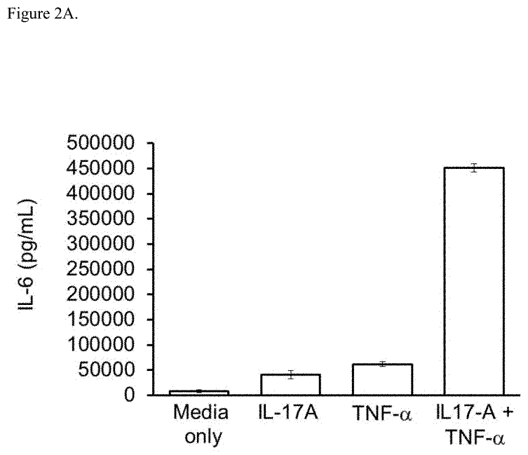

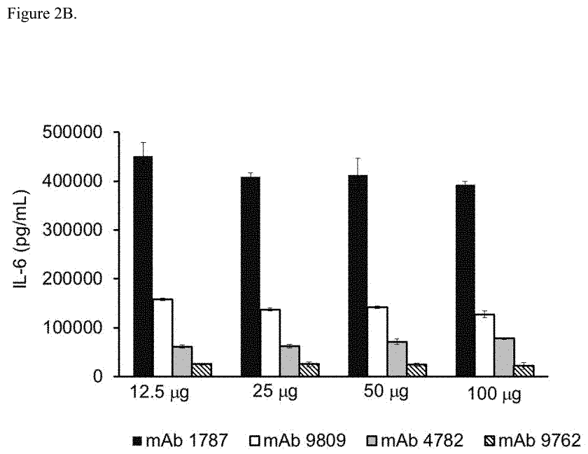

Exogenous TNF-α and IL-17A induced a 3-4 fold up-regulation of IL-6 in FLS-chondrocyte co-culture (FIG. 2A). The bispecific mAb 9762 effectively inhibited cytokine activity in the presence of chondrocytes, similar to what was observed with FLS cultures alone, wherein the effect was less with the parental anti-TNF-α (mAb 9809) or the parental anti-IL-17A (mAb 4782) antibody (FIG. 2B).

The data from the PCR arrays showed that the combination of TNF-α and IL-17A induced an apparent additive upregulation of gene expression of 13 out of the 84 inflammatory genes tested in cultures of FLS. The upregulated genes were CCL1, CCL3, CCL4, CCL7, CCL8, CCL9, CCL20, CSF2, CSF3, CXCL1, CXCL3, CXCL6 and interferon gamma, with changes observed between 13 and 20,000 fold. Additive upregulation was defined as fold changes greater than 3 times that of IL-17A only and TNF-α only treated FLS cultures. Additionally, mAb 9762 treatment prevented the additive up-regulation of all 13 genes. Confirmatory RT-PCR analysis was performed on the same experimental groups utilized in the above PCR array experiment, including co-cultures treated with mAb 9762, mAb 9809 (parental anti-TNF-α mAb), mAb 4782 (parental anti-IL-17A mAb) and isotype control. Similar additive gene expression acceptance criterion utilized in the above experiment was applied here. The RT-PCR data confirmed that TNF-α and IL-17A treatment led to increases in gene expression, while mAb 9762 inhibited the expression of these genes at all concentrations evaluated. These studies demonstrated clear activity of mAb 9762 on responses induced by TNF-α and IL-17A in a FLS-chondrocyte co-culture system. These responses were consistent to those observed with the FLS monocultures described above. The co-culture experiments indicated that chondrocytes may impact the individual gene expression patterns. However, the overall trend suggested that this is largely a FLS driven response and that the chondrocytes may only have limited effects on the response of TNF-α and IL-17A, and consequently, on the inhibitory activity of mAb 9762.

Neutralization of Inflammatory Responses Mediated by Human Native TNF-α and IL-17A Combination in RA-FLS and Activated Tₕ17/Tₕl Cell Co-Culture

The cross-talk between Tₕ17 and synoviocytes may represent the immunopathogenic basis of rheumatoid synovitis as a chronic disease, since it would result in the enduring reciprocal activation of these cells, along with recruitment of neutrophils and peripheral Tₕ17 cells, and progressive articular damage. The ability of the bispecific anti-TNF-α/IL-17A mAb 9762 to neutralize inflammatory responses mediated by endogenous TNF-α and IL-17A in human cellular system modeling in vivo complexity and cross-talk between Tₕ17 cells and synoviocytes was tested using co-cultures of RA-FLS and Tₕ17/Th₁ cells. In this co-culture system IL-17A and TN-α were endogenously produced by activated T-cells and induced inflammatory cytokine release from RA-FLS.

Briefly, RA-FLS (Articular Engineering) cells were seeded into a 96-well flat bottom tissue culture plate at 5,000 cells per well in CMRL media (Lonza) and incubated overnight (37°, 5% CO₂). Following incubation, mixed population of Tₕ1/Tₕ17 cells were added to a final concentration of 20,000 cells per well in the presence of pre-incubated dilution series of antibodies (30-0.0015 μg/mL) mAbs 9762, 4782, 9809 or a combination of 9809 and 4782 or irrelevant IgG1 isotype control antibody mAb 1787. Th1/TH17 cells alone with no antibody added were included as controls, while samples consisting of culture medium only were included as negative controls. Co-cultures were incubated for 24 h (37°, 5% CO2) and culture supernatants were collected and assayed by ELISA for IL-6 and GROα using human Duo Sets (R&D Systems, Inc.) according to manufacturer's instructions.

The parental anti-TNF-α and anti-IL-17A antibodies mAb 9809 and mAb 4782 only partially neutralized IL-6 and GROα release in RA-FLS +Tₕ17/Tₕ1 α-culture system, suggesting that both TNF-α and IL-17A contributed to inflammatory responses and neutralization of either cytokine alone may not provide adequate regulation of inflammatory responses. The bispecific anti-TNF-α/IL-17A antibody mAb 9762 was able to inhibit IL-6 and GROα production from co-culture in dose-dependent manner similarly to combination of parental anti-TNF-α and anti-IL-17A antibodies. Table 24 shows the maximum percent neutralization of IL-6 and GROα release calculated separately for each independent experiments for the mAb 9762 and the parental anti-TNF-α and anti-IL-17A mAbs either alone or in combination.

TABLE 24

| | | | | |
|---|---|---|---|---|
| Experiment | mAb 4782 | mAb 9809 | mAb 9762 | mAb 4782 + mAb 9809 |

GRO release (% Inhibition at highest Mab Concentration)

| | | | | |
|---|---|---|---|---|
| 1 | 95.2^{NS} | 60.1**** | 98.7 | NA |
| 2 | 73.1**** | 78.2**** | 97.8 | 97.2 |
| 3 | 74.6**** | 68.2**** | 96 | 95.5 |
| 4 | 82.2** | 67.9*** | 96.9 | 96.8 |
| 5 | 84.5**** | 77.5**** | 98.5 | 97.5 |

IL6 release (% Inhibition at highest Mab Concentration

| | | | | |
|---|---|---|---|---|
| 1 | 72.1*** | 54.8**** | 85.7 | NA |
| 2 | 62**** | 68.3**** | 86.5 | 86 |
| 3 | 64.3**** | 65.7**** | 86.1 | 83.4 |
| 4 | 61.4*** | 54.8**** | 69.3 | 67.5 |
| 5 | 51.6**** | 56.7*** | 77.9 | 73.1 |

Comparison of raw data (not % inhibition) for statistical significance.
One Way ANOVA with Tukey Post test, comparison of CNTO 4782 or CNTO 9809 vs CNTO 9762: ns = not significant, * = p < 0.05, ** = p < 0.01, *** = p < 0.001, **** = p < 0.0001.
NA: not done

Neutralization of rhIL17-Induced Neutrophil Influx in the Mouse Lung

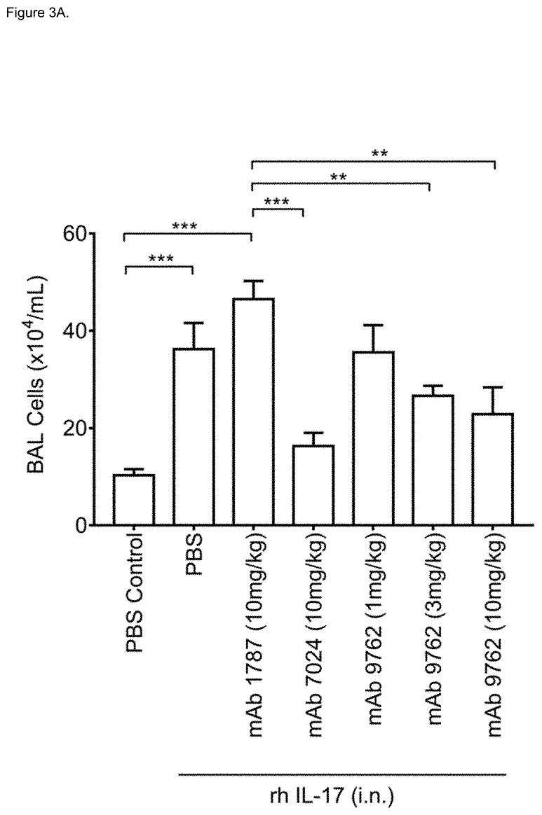

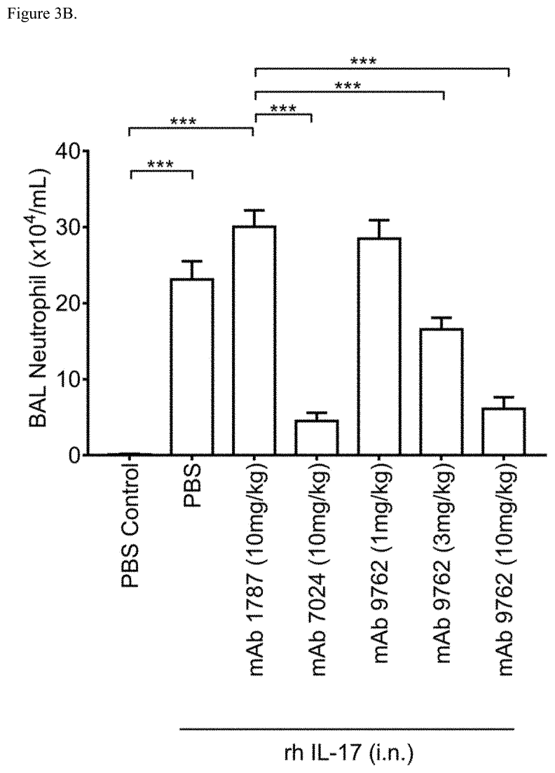

To evaluate in vivo target engagement, neutralization of recombinant human IL-17A mediated inflammatory responses was assessed in acute pharmacodynamics mouse models. Briefly, male BALB/c mice (6 to 8 weeks old) were dosed with anti-IL-17A (mAb 7024) or bispecific anti-TNF-α/IL-17A antibody (mAb 9762) intraperitoneally, 24 hours prior to intranasal rhIL-17A challenge. After 6 h, their lungs were lavaged with two volumes of 0.7 ml cold PBS containing 0.1% BSA. Total and differential cells were counted manually mAb 1787 was used as an isotype control. Intranasal rhIL-17A challenge caused robust cellular response characterized by a dominant neutrophilia in airway lumen of mice. Treatment with anti-IL-17A or bispecific anti-TNF-α/IL-17A antibody resulted in significant inhibition in rhIL-17A induced total cell influx into BAL (FIG. 3A) as well as the neutrophil influx (FIG. 3B). Data are presented as mean ±SEM; N-7 mice/group in the Figures. Asterisks (*) denote significance (*p<0.05; **p<0.01; ***p<0.001; ****p<0.0001; ANOVA followed by post-hoc test. These data indicated that bispecific anti-TNF-α/IL-17A antibody is able to neutralize human IL-17A mediated in vivo responses in dose-dependent manner

Neutralization of Lung Neutrophilia Induced by Combined Intranasal Administration of rhlL-17 and TNF-α

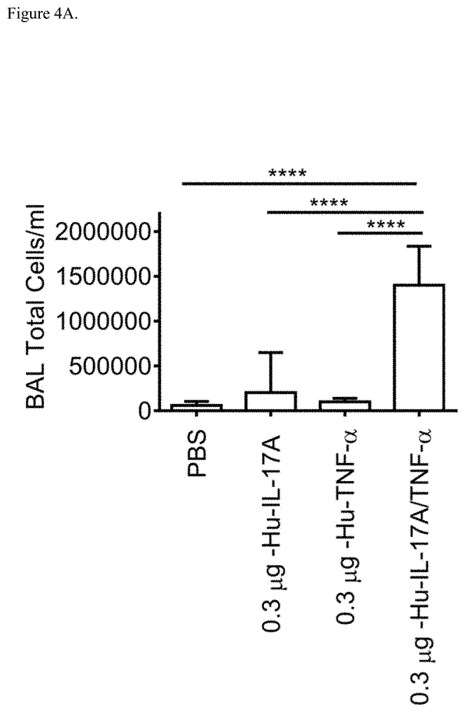

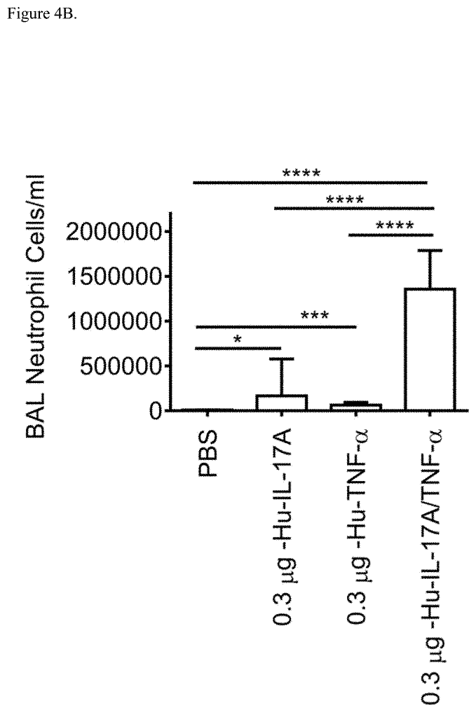

To measure neutralization of both TNF-α and IL-17A inflammatory responses, in vivo acute pharmacodynamics model was developed when mice were challenged with combination of human recombinant cytokines. Briefly, rhTNF-α (0.3 μg) and IL-17A (0.3 μg) were instilled into the lungs of Balb/c mice. After six hours, bronchoalveolar lavage was performed on the mice, and total cell influx, as well as neutrophil influx, was assessed. While a significant effect was observed with rhIL-17A instillation, a robust influx of total cells (FIG. 4A) and neutrophils (FIG. 4B) was observed with the combination of rhTNF-α and rhlL-17A. In the FIG. 4A and FIG. 4B, data represent mean+SE. Asterisks (*) denote significance (*p<0.05; **p<0.01; ***p<0.001; ****p<0.0001; ANOVA followed by post-hoc test.

Next, Balb/c mice were dosed with antibodies mAb 1787 (isotype control) mAb 4782 (anti-IL-17A antibody), mAb 9809 (anti-TNF-α antibody) or mAb 9762 (bispecific anti-TNF-α/IL-17A antibody) for 18 hours before intranasal instillation of 0.3 μg recombinant human TNF-α and IL-17A in combination Animals were euthanized 6 hours post i.n. cytokine instillation and lungs were lavaged and cell influx assessed by Advia and cytospin. The levels of pro-inflammatory cytokines were measured in BAL and serum samples.

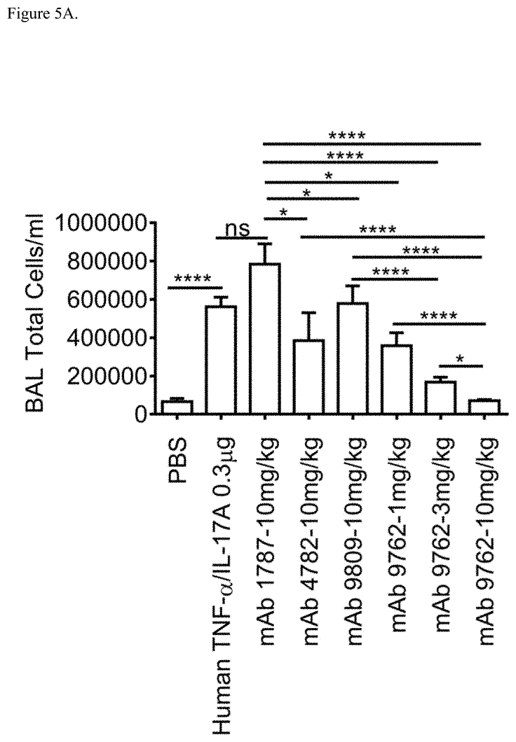

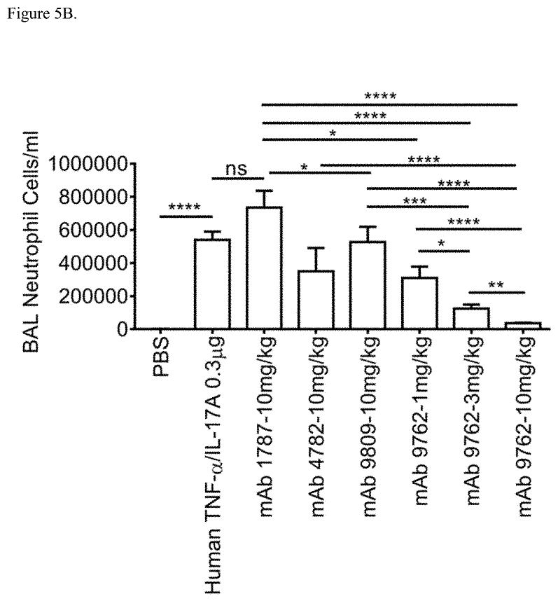

While anti-TNF-α and anti-IL-17A monoclonal antibodies, dosed at 10 mg/kg, partially attenuated cell accumulation in the lung, treatment of the mice with the bispecific anti-TNF-α/IL-17A antibody at 1 mg/kg, 3 mg/kg, and 10 mg/kg resulted in a significant, dose-dependent inhibition of cell influx, with a near complete ablation of total cells (FIG. 5A) or neutrophils (FIG. 5B) in the lung with the highest dose of 10 mg/kg. In FIG. 5A and FIG. 5B, data represent mean +SE. Data were transformed to log data and statistical analysis performed using one-way ANOVA/Tukey's Multiple Comparison Test. Asterisks (*) denote significance (*p<0.05; **p<0.01; ***p<0.001; ****p<0.0001; ANOVA followed by post-hoc test.

In addition to cell influx the level of inflammatory cytokines in BALF and serum samples was assessed. Of the 32 analytes, G-CSF, IL-6, IP10, KC, and MCP-1 were found to be elevated in BALF and serum samples following combined TNF-α and IL-17A intranasal challenge, and LIX was significantly elevated in BALF after challenge. Bispecific anti-TNF-α/IL-17A antibody treatment significantly decreased pro-inflammatory cytokine levels in BALF samples to a greater extent than a similar dose of anti-IL-17A for all six cytokines reported (p<0.05, one-way ANOVA), and significantly decreased pro-inflammatory cytokine levels in BALF compared to anti-TNF-α alone for G-CSF, KC, and LIX (p<0.05, one-way ANOVA). These data demonstrated superior inhibition of TNF-α+IL-17A mediated inflammatory responses by bispecific anti-TNF-α/IL-17A antibody relative to the monospecific anti-TNF-α or anti-IL-17A antibodies at similar dose. These data confirmed functional activity of bispecific anti-TNF-α/IL-17A antibody toward neutralization of both ligands in in vivo acute pharmacodynamic models.

We claim:

1) An isolated bispecific anti-tumor necrosis factor (TNF-α)/interleukin-17A (IL-17A) antibody comprising a first domain specifically binding TNF-α and a second domain specifically binding IL-17A, wherein the first domain comprises a heavy chain complementarity determining region (HCDR) 1, a HCDR2, a HCDR3, a light chain complementarity determining region (LCDR) 1, a LCDR2 and a LCDR3 of SEQ ID NOs: 15, 16, 17, 18, 19 and 20, respectively, and the second domain comprises the HCDR1, the HCDR2, the HCDR3, the LCDR1, the LCDR2 and the LCDR3 of SEQ ID NOs: 21, 22, 23, 24, 25 and 26, respectively.

2) The bispecific anti-TNF-α/IL-17A antibody of claim 1, wherein the antibody has one or two of the following properties:
a) inhibits IL-17A/F-mediated IL-6 production by normal human dermal fibroblasts with an IC₅₀ value of between about 0.05 μg/ml and about 0.3 μg/ml and recombinant human TNF-α-mediated cytotoxicity in KYM-1D4 human rhabdomyosarcoma cell line cells with an IC₅₀ value of between about 0.02 nM and about 0.2 nM; and
b) binds TNF-α and IL-17A with an equilibrium dissociation constant (K_{D}) of less than about 3×10⁻¹¹ M and less than about 5×10⁻¹¹ M, respectively, when the K_{D} is measured using Biacore 3000 system at 25° C. in PBS containing 0.01% polysorbate 20 (PS-20) and 100 μg/ml bovine serum albumin.

3) The bispecific anti-TNF-α/IL-17A antibody of claim 2, wherein the first domain comprises a heavy chain variable region (VH) and a light chain variable region (VL) of SEQ ID NOs: 11 and 12, respectively.

4) The bispecific anti-TNF-α/IL-17A antibody of claim 3, wherein the second domain comprises the VH and the VL of SEQ ID NOs: 13 and 14, respectively.

5) The bispecific anti-TNF-α/IL-17A antibody of claim 4, wherein the antibody is an IgG1, an IgG2, an IgG3 or an IgG4 isotype.

6) The antibody of claim 5, comprising a K409R substitution in a first heavy chain (HC1) and a F405L substitution in a second heavy chain (HC2), wherein residue numbering is according to the EU Index.

7) The bispecific anti-TNF-α/IL-17A antibody of claim 6, further comprising M252Y, S254T and T256E substitutions in the HC1, the HC2 or the HC1 and the HC2.

8) The bispecific anti-TNF-α/IL-17A antibody of claim 7, wherein the antibody is human or humanized.

9) The bispecific anti-TNF-α/IL-17A antibody of claim 6, comprising the HC1 of SEQ ID NO: 5 and the HC2 of SEQ ID NO: 8 or the HC1 of SEQ ID NO: 7 and the HC2 of SEQ ID NO: 10.

10) The bispecific anti-TNF-α/IL-17A antibody of claim 9, comprising a first light chain (LC1) of SEQ ID NO: 6 and a second light chain (LC2) of SEQ ID NO: 9.

11) An isolated bispecific anti-TNF-α/IL-17A antibody comprising
a) the HC1 and the LC1 of SEQ ID NOs: 5 and 6, respectively, and the HC2 and the LC2 of SEQ ID NOs: 8 and 9, respectively; or
b) the HC1 and the LC1 of SEQ ID NOs: 7 and 6, respectively, and the HC2 and the LC2 of SEQ ID NOs: 10 and 9, respectively.

12) A pharmaceutical composition comprising the bispecific anti-TNF-α/IL-17A antibody of claim 1.

13) A pharmaceutical composition comprising the bispecific anti-TNF-α/IL-17A antibody of claim 11.

14) The pharmaceutical composition of claim 13, comprising 40 mg/ml of the bispecific anti-TNF-α/IL-17A antibody, 10 mM histidine, 8.5% (w/v) sucrose and 0.04% (w/v) Polysorbate 80 at pH 5.8.

15) A method of treating a TNF-α- and/or an IL-17A-mediated inflammatory disease, comprising administering to a subject in need thereof a therapeutically effective amount of the bispecific anti-TNF-α/IL-17A antibody of claim 11 for a time sufficient to treat the TNF-α- and/or the IL-17A-mediated inflammatory disease.

16) The method of claim 15, wherein the TNF-α- and/or the IL-17A-mediated inflammatory disease is an autoimmune disease, inflammatory bowel disease, Crohn's disease, ulcerative colitis, arthritis, rheumatoid arthritis, psoriatic arthritis, juvenile idiopathic arthritis, ankylosing spondylitis, spondyloarthritis, psoriasis, juvenile psoriasis, axial Bechet's disease, Hidradentis suppurativa, uveitis, asthma, sepsis, lupus, lupus erythematosus, cutaneous infection, cachexia, Wegener's granulomatosis, pulmonary fibrosis, chronic obstructive pulmonary disease, heart failure, Kawasaki disease, fascular sarcoidosis, type 1 diabetes, ischemia, infarction, anal fistula, ichthyosis, seborrhea or acne.

17) The method of claim 16, wherein the TNF-α- and/or the IL-17A-mediated inflammatory disease is rheumatoid arthritis (RA).

18) The method of claim 16, wherein the TNF-α- and/or the IL-17A-mediated inflammatory disease is psoriatic arthritis.

19) The method of claim 15, wherein the bispecific anti-TNF-α/IL-17A antibody is administered in combination with a second therapeutic agent.

20) The method of claim 19, wherein the second therapeutic agent is a standard of care treatment of the TNF-α- and/or the IL-17A-mediated inflammatory disease.

21) An anti-idiotypic antibody specifically binding the bispecific anti-TNF-α/IL-17A antibody of claim 11.

22) A kit comprising the bispecific anti-TNF-α/IL-17A antibody of claim 11.

23) An isolated synthetic polynucleotide
a) encoding the HC1, the LC1, the HC2 and/or the LC2 of claim 11; or
b) comprising a polynucleotide sequence of SEQ ID NOs: 33, 34, 35, 36, 37 or 38.

24) A vector comprising the polynucleotide of claim 23.

25) A host cell comprising the vector of claim 24.

26) A method of producing the isolated bispecific anti-TNF-α/IL-17A antibody of claim 11, comprising:
a) combining an isolated monospecific bivalent anti-TNF-α antibody comprising two heavy chains of SEQ ID NO: 5 or two heavy chains of SEQ ID NO: 7 and two light chains of SEQ ID NO: 6 and an isolated monospecific bivalent anti-IL-17A antibody comprising two heavy chains of SEQ ID NO: 8 or two heavy chains of SEQ ID NO: 10 and two light chains of SEQ ID NO: 9 in a mixture of about 1:1 molar ratio;
b) introducing a reducing agent into the mixture;
c) incubating the mixture about ninety minutes to about six hours;
d) removing the reducing agent; and
e) purifying
i) the bispecific anti-TNF-α/IL-17A antibody that comprises a first heavy chain of SEQ ID NO: 5 and a second heavy chain of SEQ ID NO: 8, a first light chain of SEQ ID NO: 6 and a second light chain of SEQ ID NO: 9, wherein the first heavy chain of SEQ ID NO: 5 pairs with the first light chain of SEQ ID NO: 6 to form a first binding domain that specifically binds TNF-α, and the second heavy chain of SEQ ID NO: 8 pairs with the second light chain of SEQ ID NO: 9 to form a second binding domain that specifically binds IL-17A; or
ii) purifying the bispecific anti-TNF-α/IL-17A antibody that comprises a first heavy chain of SEQ ID NO: 7 and a second heavy chain of SEQ ID NO: 10, a first light chain of SEQ ID NO: 6 and a second light chain of SEQ ID NO: 9, wherein the first heavy chain of SEQ ID NO: 7 pairs with the first light chain of SEQ ID NO: 6 to form the first binding domain that specifically binds TNF-α, and the second heavy chain of SEQ ID NO: 10 pairs with the second light chain of SEQ ID NO: 9 to form the second binding domain that specifically binds IL-17A.

27) The method of claim 26, wherein the reducing agent is 2-mercaptoethanolamine (2-MEA).

28) The method of claim 27, wherein the 2-MEA is present at a concentration of about 25 mM to about 75 mM.

29) The method of claim 28, wherein the incubating step is performed at a temperature of about 25° C. to about 37° C.

30) An isolated anti-TNF-α antibody comprising the VH of SEQ ID NO: 11 and the VL of SEQ ID NO: 12.

31) A pharmaceutical composition comprising the anti-TNF-α antibody of claim 30 and a pharmaceutically acceptable excipient.

32) An isolated synthetic polynucleotide
a) encoding the VH or the VH and the VL of claim 30; or
b) comprising a polynucleotide sequence of SEQ ID NO: 39 or SEQ ID NO: 39 and SEQ ID NO: 40.

33) A vector comprising the polynucleotide of claim 32.

34) A host cell comprising the vector of claim 33.

35) A method of producing the antibody of claim 30, comprising culturing the host cell of claim 34 in conditions that the antibody is produced, and purifying the antibody.

36) A method of treating a TNF-α mediated inflammatory disease, comprising administering to a subject in need thereof the isolated antibody of claim 30 for a time sufficient to treat the TNF-α mediated disease.

37) The method of claim 36, wherein the TNF-α mediated inflammatory disease is an autoimmune disease, inflammatory bowel disease, Crohn's disease, ulcerative colitis, arthritis, rheumatoid arthritis, psoriatic arthritis, ankylosing spondylitis, spondyloarthritis, psoriasis, juvenile psoriasis, juvenile idiopathic arthritis, axial Bechet's disease, Hidradentis suppurativa, uveitis, asthma, sepsis, lupus erythematosus, cutaneous infection, cachexia, Wegener's granulomatosis, pulmonary fibrosis, chronic obstructive pulmonary disease, heart failure, Kawasaki disease, fascular sarcoidosis, type 1 diabetes, ischemia, infarction, anal fistula, ichthyosis or seborrhea.


SEQUENCE LISTING (40 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 157 aa, Homo sapiens [sequence omitted]

SEQ ID NO: 3 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 4 - protein, 133 aa, Homo sapiens [sequence omitted]

SEQ ID NO: 5 - protein, 456 aa, Artificial Sequence
  mAb 9809, mAb 9762 TNF-alpha binding heavy chain
QVQLVESGGGVVQPGRSLRLSCAASGFIFSSYAMHWVRQAPGKGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 6 - protein, 215 aa, Artificial Sequence
  mAb 9809, mAb 6696, mAb 9762, mAb 8759 TNF-alpha binding light chain
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 456 aa, Artificial Sequence
  mAb 6696, mAb 8759 TNF-alpha binding heavy chain
QVQLVESGGGVVQPGRSLRLSCAASGFIFSSYAMHWVRQAPGKGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 8 - protein, 445 aa, Artificial Sequence
  mAb 4782, mAb 9762 IL-17 binding heavy chain
QVQLLESGGGLVQPGGSLRLSCAASGFTFSSYAMSWVRQAPGKGLEWVSTISLTSGFTYYADSVKGRFTISRDNSKNTLYLQMNSLRAEDTAVYYCARQLTLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 9 - protein, 214 aa, Artificial Sequence
  mAb 4782, mAb 7206, mAb 9762, mAb 8759 IL-17A binding light chain
QSVLTQPPSVSVAPGQTARISCSGDNLGDKYANWYQQKPGQAPVLVIYDDIDRPSGIPERFSGSNSGNTATLTISGTQAEDEADYYCGSYDFFLGMIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 445 aa, Artificial Sequence
  mAb 7206, mAb 8759 IL-17A binding  heavy chain
QVQLLESGGGLVQPGGSLRLSCAASGFTFSSYAMSWVRQAPGKGLEWVSTISLTSGFTYYADSVKGRFTISRDNSKNTLYLQMNSLRAEDTAVYYCARQLTLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 11 - protein, 126 aa, Artificial Sequence
  mAb 9762, mAb 8759 TNF-alpha binding  VH
QVQLVESGGGVVQPGRSLRLSCAASGFIFSSYAMHWVRQAPGKGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 12 - protein, 108 aa, Artificial Sequence
  mAb 9762, mAb 8759 TNF-alpha binding VL
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 13 - protein, 115 aa, Artificial Sequence
  mAb9762, mAb 8759 IL-17A binding VH
QVQLLESGGGLVQPGGSLRLSCAASGFTFSSYAMSWVRQAPGKGLEWVSTISLTSGFTYYADSVKGRFTISRDNSKNTLYLQMNSLRAEDTAVYYCARQLTLDVWGQGTLVTVSS

SEQ ID NO: 14 - protein, 108 aa, Artificial Sequence
  mAb9762, mAb 8759 IL-17A binding VL
QSVLTQPPSVSVAPGQTARISCSGDNLGDKYANWYQQKPGQAPVLVIYDDIDRPSGIPERFSGSNSGNTATLTISGTQAEDEADYYCGSYDFFLGMIVFGGGTKLTVL

SEQ ID NO: 15 - protein, 5 aa, Artificial Sequence
  HCDR1 of TNF-  binding domain of mAb 9762
SYAMH

SEQ ID NO: 16 - protein, 17 aa, Artificial Sequence
  HCDR2 of TNF-  binding domain of mAb 9762
FMSYDGSNKKYADSVKG

SEQ ID NO: 17 - protein, 17 aa, Artificial Sequence
  HCDR3 of TNF-  binding domain of mAb 9762
DRGIAAGGNYYYYGMDV

SEQ ID NO: 18 - protein, 11 aa, Artificial Sequence
  LCDR1 of TNF-  binding domain of mAb 9762
RASQSVYSYLA

SEQ ID NO: 19 - protein, 7 aa, Artificial Sequence
  LCDR2 of TNF-  binding domain of mAb 9762
DASNRAT

SEQ ID NO: 20 - protein, 10 aa, Artificial Sequence
  LCDR3 of TNF-  binding domain of mAb 9762
QQRSNWPPFT

SEQ ID NO: 21 - protein, 5 aa, Artificial Sequence
  HCDR1 of IL-17A binding domain of mAb 9762
SYAMS

SEQ ID NO: 22 - protein, 17 aa, Artificial Sequence
  HCDR2 of IL-17A binding domain of mAb 9762
TISLTSGFTYYADSVKG

SEQ ID NO: 23 - protein, 5 aa, Artificial Sequence
  HCDR3 of IL-17A binding domain of mAb 9762
LTLDV

SEQ ID NO: 24 - protein, 11 aa, Artificial Sequence
  LCDR1 of IL-17A binding domain of mAb 9762
SGDNLGDKYAN

SEQ ID NO: 25 - protein, 7 aa, Artificial Sequence
  LCDR2 of IL-17A binding domain of mAb 9762
DDIDRPS

SEQ ID NO: 26 - protein, 11 aa, Artificial Sequence
  LCDR3 of IL-17A binding domain of mAb 9762
GSYDFFLGMIV

SEQ ID NO: 27 - protein, 133 aa, Mus musculus
AAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 28 - protein, 132 aa, Macaca fascicularis
GIAIPRNSGCPNSEDKNFPRTVMVNLNIHNRNTSTNPKRSSDYYNRSTSPWNLHRNEDPERYPSVIWEAKCRHLGCVKADGNVDYHMNSVPIQQEILVLRREPRHCPNSFRLEKILVSVGCTCVTPIVHHVA

SEQ ID NO: 29 - protein, 133 aa, Rattus norvegicus
AVLIPQSSVCPNAEANNFLQNVKVNLKVLNSLSSKASSRRPSDYLNRSTSPWTLSRNEDPDRYPSVIWEAQCRHQRCVNAEGKLDHHMNSVLIQQEILVLKREPEKCPFTFRVEKMLVGVGCTCVSSIVRHAS

SEQ ID NO: 30 - protein, 157 aa, Mus musculus
MLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 31 - protein, 157 aa, Macaca fascicularis
VRSSSRTPSDKPVAHVVANPQAEGQLQWLNRRANALVANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 32 - protein, 157 aa, Rattus norvegicus
MLRSSSQNSSDKPVAHVVANHQAEEQLEWLSQRANALLANGMDLKDNQLVVPADGLYLIYSQVLFKGQGCPDYVLLTHTVSRFAISYQEKVSLLSAIKSPCPKDTPEGAELKPWYEPMYLGGVFQLEKGDLLSAEVNLPKYLDITESGQVYFGVIAL

SEQ ID NO: 33 - DNA, 1368 bp, Artificial Sequence
  DNA encoding HC1 of SEQ ID NO 5
caggtacagctggtggagtctgggggaggcgtggtccagcctgggaggtccctgagactctcctgtgcagcctctggattcatcttcagtagctatgctatgcactgggtccgccaggcaccaggcaaagggctggagtgggtggcatttatgtcatatgatggaagcaataaaaagtacgcagactccgtgaagggccgattcaccatctccagagacaattccaagaacacgctgtatctgcaaatgaacagcctgagagctgaggacacggctgtgtattactgtgcgagagatcgaggaatagcagcaggtggaaactactactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - DNA, 1368 bp, Artificial Sequence
  DNA encoding HC1 of SEQ ID NO 7
caggtacagctggtggagtctgggggaggcgtggtccagcctgggaggtccctgagactctcctgtgcagcctctggattcatcttcagtagctatgctatgcactgggtccgccaggcaccaggcaaagggctggagtgggtggcatttatgtcatatgatggaagcaataaaaagtacgcagactccgtgaagggccgattcaccatctccagagacaattccaagaacacgctgtatctgcaaatgaacagcctgagagctgaggacacggctgtgtattactgtgcgagagatcgaggaatagcagcaggtggaaactactactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagagcaggtggcagcaggggaacgtcttctcatgctccgtgatgcatgaggctctgcacaaccactacacgcagaagagcctctccctgtctccgggtaaa

SEQ ID NO: 35 - DNA, 645 bp, Artificial Sequence
  DNA encoding LC of SEQ ID NO 6
gaaattgtgttgacacagtctccagccaccctgtctttgtctccaggggaaagagccaccctctcctgcagggccagtcagagtgtttacagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 36 - DNA, 1335 bp, Artificial Sequence
  DNA encoding HC of SEQ ID NO 8
caagtgcagctgctggagagcggcggcggcctggtgcagcccggcggcagcctgcggctgagctgcgccgccagcggcttcaccttcagcagctacgccatgagctgggtgcggcaggcccccggcaagggcctggagtgggtgagcaccatcagcctgaccagcggcttcacctactacgccgacagcgtgaagggccggttcaccatcagccgggacaacagcaagaacaccctgtacctgcagatgaacagcctgcgggccgaggacaccgccgtgtactactgcgcccggcagctgaccctggacgtgtggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - DNA, 1335 bp, Artificial Sequence
  DNA encoding HC of SEQ ID NO 10
caagtgcagctgctggagagcggcggcggcctggtgcagcccggcggcagcctgcggctgagctgcgccgccagcggcttcaccttcagcagctacgccatgagctgggtgcggcaggcccccggcaagggcctggagtgggtgagcaccatcagcctgaccagcggcttcacctactacgccgacagcgtgaagggccggttcaccatcagccgggacaacagcaagaacaccctgtacctgcagatgaacagcctgcgggccgaggacaccgccgtgtactactgcgcccggcagctgaccctggacgtgtggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 38 - DNA, 642 bp, Artificial Sequence
  DNA encoding LC of SEQ ID NO 9
cagagcgtgctgacccagccccccagcgtgagcgtggcccccggccagaccgcccggatcagctgcagcggcgacaacctgggcgacaagtacgccaactggtaccagcagaagcccggccaggcccccgtgctggtgatctacgacgacatcgaccggcccagcggcatccccgagcggttcagcggcagcaacagcggcaacaccgccaccctgaccatcagcggcacccaggccgaggacgaggccgactactactgcggcagctacgacttcttcctgggcatgatcgtgttcggcggcggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 39 - DNA, 378 bp, Artificial Sequence
  DNA encoding VH of anti-TNF alpha mAb
caggtacagctggtggagtctgggggaggcgtggtccagcctgggaggtccctgagactctcctgtgcagcctctggattcatcttcagtagctatgctatgcactgggtccgccaggcaccaggcaaagggctggagtgggtggcatttatgtcatatgatggaagcaataaaaagtacgcagactccgtgaagggccgattcaccatctccagagacaattccaagaacacgctgtatctgcaaatgaacagcctgagagctgaggacacggctgtgtattactgtgcgagagatcgaggaatagcagcaggtggaaactactactactacggtatggacgtctggggccaagggaccacggtcaccgtctcctca

SEQ ID NO: 40 - DNA, 324 bp, Artificial Sequence
  DNA encoding VL of anti-TNF alpha mAb
gaaattgtgttgacacagtctccagccaccctgtctttgtctccaggggaaagagccaccctctcctgcagggccagtcagagtgtttacagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccattcactttcggccctgggaccaaagtggatatcaaa